(19) United States
(12) Patent Application Publication
(10) Pub. No.: US 2006/0210978 A1
(43) Pub. Date: Sep. 21, 2006

(54) Proteome-wide mapping of post-translational modifications using endopeptidases

(75) Inventors: Kevan M. Shokat, San Francisco, CA (US); Zachary Knight, San Francisco, CA (US)

Correspondence Address: TOWNSEND AND TOWNSEND AND CREW, LLP, TWO EMBARCADERO CENTER, EIGHTH FLOOR, SAN FRANCISCO, CA, 94111-3834, US

(73) Assignee: The Regents of the University of California, Oakland, CA (US)

(21) Appl. No.: 10/524,608

(22) PCT Filed: Aug. 14, 2003
(86) PCT No.: PCT/US03/25456

§ 371 Date: Mar. 24, 2006

Related U.S. Application Data
(60) Provisional application No. 60/405,589, filed on Aug. 14, 2002.

(51) Int. Cl. C12Q 1/68; C12Q 1/37; C07H 21/04; C12P 21/06; C12N 9/64

(52) U.S. Cl. 435/6; 435/23; 435/69.1; 435/226; 435/320.1; 435/325; 536/23.2

(57) ABSTRACT

The present invention provides novel endonucleases that site-specifically cleave a post-translationally modified polypeptide at a site of post-translational modification. The present invention further provides methods making and using the endonucleases, including methods of mapping post-translational modifications in the human genome.

### CROSS-REFERENCES TO RELATED APPLICATIONS

The present application claims priority to U.S. Provisional Patent Application No. 60/405,589, filed Aug. 14, 2002, the disclosure of which is incorporated herein in its entirety for all purposes.

### STATEMENT AS TO RIGHTS TO INVENTIONS MADE UNDER FEDERALLY SPONSORED RESEARCH AND DEVELOPMENT

The present invention was supported by a grant from the National Institutes of Health (CA 70031). The Government may have rights in this invention.

### BACKGROUND OF THE INVENTION

Protein post-translational modification is one of the dominant mechanisms of information transfer in cells. A major goal of current proteomic efforts is to generate a system level map describing all the sites of protein post-translational modification. Recent effort toward this goal has focused on developing new technologies for enriching and quantitating phosphopeptides. By contrast, identification of the sites of phosphorylation typically relies exclusively on the use of tandem mass spectrometry to sequence individual peptides.

Much of the complexity of higher organisms is believed to reside in the specific post-translational modification of proteins (Venter et al., Science, 2001, 291(5507): 1304-51.). Protein phosphorylation is the most ubiquitous such modification; almost 2% of the human genome encodes protein kinases and an estimated one-third of all proteins contain a covalently bound phosphate group (Manning et al., Science, 2002, 298(5600): 1912-34). Due to the importance of protein phosphorylation in regulating cellular signaling events, there is intense interest in developing technologies for mapping phosphorylation events on a proteome-wide scale.

Existing approaches for phosphorylation site mapping rely almost exclusively on the use of tandem mass spectrometry (MS/MS) to sequence individual peptides in order to localize sites of phosphorylation. Despite the power of this approach, MS/MS of phosphopeptides remains challenging due to (i) the signal suppression of phosphate containing molecules in the commonly used positive detection mode, (ii) the difficulty in achieving full sequence coverage, especially for long peptides, peptides present in low abundance, and peptides phosphorylated at sub-stoichiometric levels—all of which are common for phosphopeptides, (iii) the difficulty in localizing the phosphoamino acid within an MS/MS spectrum due to the inherent lability of the phosphate group, and (iv) the inability to distinguish between distinct phosphoisoforms of a single polypeptide that may coexist in a biological sample (McLachlin et al., Curr Opin Chem Biol, 2001, 5(5): 591-602; Mann et al., Trends Biotechnol, 2002, 20(6): 261-8; Zhou et al., Nat Biotechnol, 2001, 19(4): 375-8; Oda et al., Nat Biotechnol, 2001, 19(4): 379-82; Steen et al., J Am Soc Mass Spectrom, 2002, 13(8): p. 996-1003). The challenge of mapping phosphorylation sites is highlighted by recent efforts to enrich phosphopeptides from complex mixtures. While these strategies have provided powerful tools for purifying phosphopeptides, the next step—identifying the precise site of phosphorylation—often fails for many of the peptides that are recovered.

Currently, the first step in mapping the phosphorylation sites of a protein is to digest the phosphoprotein with a protease (e.g., trypsin) that generates smaller peptide fragments for sequencing. We reasoned that this process would be more informative if a protease that specifically cleaved its substrates at the site of phosphorylation were used. Such a digestion would selectively hydrolyze the amide bond adjacent to each phosphorylated residue, facilitating identification of the phosphorylation site directly from the cleavage pattern (e.g., from an MS ‘fingerprint’ specifying the exact masses of the cleavage products). Phosphospecific cleavage would also facilitate the interpretation of MS/MS spectra, since the C-terminal residue would always be the formerly phosphorylated residue, resulting in a unique y₁ ion. In this regard, it is often possible to obtain tandem mass spectra of a phosphopeptide, but still fail to localize the phosphoamino acid within that sequence. Presently, no protease is known that selectively recognizes a phosphorylated amino acid, or any other post-translational modification.

A method to address this problem utilizing a strategy for specific proteolysis at sites of post-translational modification, such as phosphorylation, would represent a significant advance in the art. The present invention satisfies this and other needs.

### BRIEF SUMMARY OF THE INVENTION

The present invention provides novel endonucleases for use in mapping post-translational modification sites in a genome, such as the human genome. The present invention provides endonucleases that, surprisingly, site-specifically cleave a post-translationally modified polypeptide at a site of post-translational modification.

In a first aspect, the invention provides a method of mapping the sites of polypeptide post-translational modifications. The method includes site-specifically cleaving a peptide bond of the post-translationally modified polypeptide with an endopeptidase at a site of post-translational modification to produce a degraded post-translationally modified polypeptide. After cleavage at the site of post-translational modification, the site of post-translational modification is determined.

In another aspect, the present invention provides an endopeptidase that site-specifically cleaves a peptide bond of a post-translationally modified polypeptide at a site of post-translational modification, wherein the endopeptidase comprises an active site that binds to said post-translational modification.

In another aspect, the endopeptidases of the present invention are produced by a method that includes introducing one or more point mutations into a model endopeptidase at one or more candidate amino acid positions in an active site of the model endopeptidase to produce a plurality of candidate endopeptidases. At least one of the plurality of the candidate endopeptidases is an endopeptidases of the present invention that site-specifically cleaves a peptide bond of a post-translationally modified polypeptide at a site of post-translational modification. The endopeptidase that site-specifically cleaves at said site of post-translational modification is identified by contacting each of the plurality of candidate endopeptidases with the post-translationally modified polypeptide to determine whether or not each candidate endopeptidase site-specifically cleaves the peptide bond of the polypeptide at the site of a post-translational modification.

In another aspect, the present invention provides an isolated nucleic acid encoding a endopeptidase which site-specifically cleaves a peptide bond of a post-translationally modified polypeptide at a site of post-translational modification and which comprises one or more point mutations at one or more amino acid positions within the endopeptidase active site. The isolated nucleic acid contains a subsequence having at least 70% nucleic acid sequence identity to a nucleic acid sequence of FIG. 2.

In another aspect, the present invention provides an isolated nucleic acid encoding a endopeptidase which site-specifically cleaves a peptide bond of a post-translationally modified polypeptide at a site of post-translational modification and which comprises one or more point mutations at one or more amino acid positions within the endopeptidase active site. The isolated nucleic acid hybridizes under highly stringent hybridization conditions to a nucleic acid sequence of FIG. 2, wherein the hybridization reaction is incubated at 42° C. in a solution comprising 50% formamide, 5×SSC and 1% SDS, and washed at 65° C. in a solution comprising 0.2×SSC and 0.1% SDS.

### BRIEF DESCRIPTION OF THE DRAWINGS

FIG. 1 is an amino acid sequence of a subtilisin model endopeptidase.

FIG. 2 is a nucleic acid sequence that encodes a subtilisin model endopeptidase.

FIG. 3 illustrates a comparison of a computer generated three-dimensional structure of the model subtilisin and a phosphotyrosine polypeptide.

FIG. 4 illustrates the phosphotyrosine site-specificity of candidate subtilisin endopeptidases and the model subtilisin endopeptidase against either an unmodified tyrosine or phenylalanine.

FIG. 5 shows kinetic data for the site-specific cleavage at a phosphotyrosine by a subtilisin endopeptidase containing the substitution point mutations P129G and E156R.

FIG. 6 shows kinetic data for the site-specific cleavage at a phosphotyrosine by a subtilisin endopeptidase containing the substitution point mutations G127S and E156R.

FIG. 7 is an amino acid sequence of a subtilisin model endopeptidase containing a signal sequence (in bold) and a pro-domain (underlined).

FIG. 8 is a nucleic acid sequence that encodes a subtilisin model endopeptidase containing a signal sequence (in bold) and a pro-domain (underlined).

### DETAILED DESCRIPTION OF THE INVENTION

In contrast to presently utilized methods of developing a system level map describing all the sites of post-translational peptide modification, e.g., polypeptide phosphorylation, the present invention provides an approach for post-translational modification mapping that makes it possible to enzymatically interrogate a protein sequence directly to identify sites of post-translational modification.

Definitions

The term “point mutation” refers to a deletion, addition, or substitution at a designed amino acid position in an amino acid or nucleotide sequence. Preferably, the term refers to an amino acid substitution.

“Candidate amino acid position” refers to an amino acid position in the active site of a model endopeptidase that is selected for deletion or substitution of the amino acid at the position or for addition of an amino acid at the position. The selection of the candidate amino acid position may be at random or rationally based. Preferably, the selection is rationally based on a comparison between three-dimensional structures of the model endopeptidase active site and the post-transitionally modified polypeptide.

“Nucleic acid” refers to deoxyribonucleotides or ribonucleotides and polymers thereof in single- or double-stranded form, or complements thereof. The term encompasses nucleic acids containing known nucleotide analogs or modified backbone residues or linkages, which are synthetic, naturally occurring, and non-naturally occurring, which have similar binding properties as the reference nucleic acid, and which are metabolized in a manner similar to the reference nucleotides. Examples of such analogs include, without limitation, phosphorothioates, phosphoramidates, methyl phosphonates, chiral-methyl phosphonates, 2-O-methyl ribonucleotides, peptide-nucleic acids (PNAs). Nucleic acids also include complementary nucleic acids.

Unless otherwise indicated, a particular nucleic acid sequence also implicitly encompasses conservatively modified variants thereof (e.g., degenerate codon substitutions) and complementary sequences, as well as the sequence explicitly indicated. Specifically, degenerate codon substitutions may be achieved by generating sequences in which the third position of one or more selected (or all) codons is substituted with mixed-base and/or deoxyinosine residues (Batzer et al., Nucleic Acid Res. 19:5081 (1991); Ohtsuka et al., J. Biol. Chem. 260:2605-2608 (1985); Rossolini et al., Mol. Cell. Probes 8:91-98 (1994)). The term nucleic acid is used interchangeably with gene, cDNA, mRNA, oligonucleotide, and polynucleotide.

A particular nucleic acid sequence also implicitly encompasses “splice variants.” Similarly, a particular protein encoded by a nucleic acid implicitly encompasses any protein encoded by a splice variant of that nucleic acid. “Splice variants,” as the name suggests, are products of alternative splicing of a gene. After transcription, an initial nucleic acid transcript may be spliced such that different (alternate) nucleic acid splice products encode different polypeptides. Mechanisms for the production of splice variants vary, but include alternate splicing of exons. Alternate polypeptides derived from the same nucleic acid by read-through transcription are also encompassed by this definition. Any products of a splicing reaction, including recombinant forms of the splice products, are included in this definition.

“Conservatively modified variants” applies to both amino acid and nucleic acid sequences. With respect to particular nucleic acid sequences, conservatively modified variants refers to those nucleic acids which encode identical or essentially identical amino acid sequences, or where the nucleic acid does not encode an amino acid sequence, to essentially identical sequences. Because of the degeneracy of the genetic code, a large number of functionally identical nucleic acids encode any given protein. For instance, the codons GCA, GCC, GCG and GCU all encode the amino acid alanine. Thus, at every position where an alanine is specified by a codon, the codon can be altered to any of the corresponding codons described without altering the encoded polypeptide. Such nucleic acid variations are “silent variations,” which are one species of conservatively modified variations. Every nucleic acid sequence herein which encodes a polypeptide also describes every possible silent variation of the nucleic acid. One of skill will recognize that each codon in a nucleic acid (except AUG, which is ordinarily the only codon for methionine, and TGG, which is ordinarily the only codon for tryptophan) can be modified to yield a functionally identical molecule. Accordingly, each silent variation of a nucleic acid which encodes a polypeptide is implicit in each described sequence with respect to the expression product, but not with respect to actual probe sequences.

As to amino acid sequences, one of skill will recognize that individual substitutions, deletions or additions to a nucleic acid, peptide, polypeptide, or protein sequence which alters, adds or deletes a single amino acid or a small percentage of amino acids in the encoded sequence is a “conservatively modified variant” where the alteration results in the substitution of an amino acid with a chemically similar amino acid. Conservative substitution tables providing functionally similar amino acids are well known in the art. Such conservatively modified variants are in addition to and do not exclude polymorphic variants, interspecies homologs, and alleles of the invention.

The following eight groups each contain amino acids that are conservative substitutions for one another:

1) Alanine (A), Glycine (G);

2) Aspartic acid (D), Glutamic acid (E);

3) Asparagine (N), Glutamine (Q);

4) Arginine (R), Lysine (K);

5) Isoleucine (I), Leucine (L), Methionine (M), Valine (V);

6) Phenylalanine (F), Tyrosine (Y), Tryptophan (W);

7) Serine (S), Threonine (T); and

8) Cysteine (C), Methionine (M)

(see, e.g., Creighton, Proteins (1984)).

Macromolecular structures such as polypeptide structures can be described in terms of various levels of organization. For a general discussion of this organization, see, e.g., Alberts et al., Molecular Biology of the Cell (3^{rd} ed., 1994) and Cantor and Schimmel, Biophysical Chemistry Part I: The Conformation of Biological Macromolecules (1980). “Primary structure” refers to the amino acid sequence of a particular peptide. “Secondary structure” refers to locally ordered, three dimensional structures within a polypeptide. These structures are commonly known as domains. Domains are portions of a polypeptide that form a compact unit of the polypeptide and are typically about 18 to 350 amino acids long, e.g., the transmembrane regions, pore loop domain, and the C-terminal tail domain. Typical domains are made up of sections of lesser organization such as stretches of β-sheet and α-helices. “Tertiary structure” refers to the complete three dimensional structure of a polypeptide monomer. “Quaternary structure” refers to the three dimensional structure formed by the noncovalent association of independent tertiary units. Anisotropic terms are also known as energy terms.

The term “recombinant” when used with reference, e.g., to a cell, or nucleic acid, protein, or vector, indicates that the cell, nucleic acid, protein or vector, has been modified by the introduction of a heterologous nucleic acid or protein or the alteration of a native nucleic acid or protein, or that the cell is derived from a cell so modified. Thus, for example, recombinant cells express genes that are not found within the native (non-recombinant) form of the cell or express native genes that are otherwise abnormally expressed, under expressed or not expressed at all.

An “expression vector” is a nucleic acid construct, generated recombinantly or synthetically, with a series of specified nucleic acid elements that permit transcription of a particular nucleic acid in a host cell. The expression vector can be part of a plasmid, virus, or nucleic acid fragment. Typically, the expression vector includes a nucleic acid to be transcribed operably linked to a promoter.

The terms “identical” or percent “identity,” in the context of two or more nucleic acids or polypeptide sequences, refer to two or more sequences or subsequences that are the same or have a specified percentage of amino acid residues or nucleotides that are the same (i.e., 60% identity, preferably 65%, 70%, 75%, 80%, 85%, 90%, or 95% identity over a specified region), when compared and aligned for maximum correspondence over a comparison window, or designated region as measured using one of the following sequence comparison algorithms or by manual alignment and visual inspection. Such sequences are then said to be “substantially identical.” This definition also refers to the compliment of a test sequence. Preferably, the identity exists over a region that is at least about 25 amino acids or nucleotides in length, or more preferably over a region that is 50-100 amino acids or nucleotides in length.

For sequence comparison, typically one sequence acts as a reference sequence, to which test sequences are compared. When using a sequence comparison algorithm, test and reference sequences are entered into a computer, subsequence coordinates are designated, if necessary, and sequence algorithm program parameters are designated. Default program parameters can be used, or alternative parameters can be designated. The sequence comparison algorithm then calculates the percent sequence identities for the test sequences relative to the reference sequence, based on the program parameters. For sequence comparison of nucleic acids and proteins, the BLAST and BLAST 2.0 algorithms and the default parameters discussed below are used.

A “comparison window,” as used herein, includes reference to a segment of any one of the number of contiguous positions selected from the group consisting of from 20 to 600, usually about 50 to about 200, more usually about 100 to about 150 in which a sequence may be compared to a reference sequence of the same number of contiguous positions after the two sequences are optimally aligned. Methods of alignment of sequences for comparison are well-known in the art. Optimal alignment of sequences for comparison can be conducted, e.g., by the local homology algorithm of Smith & Waterman, Adv. Appl. Math. 2:482 (1981), by the homology alignment algorithm of Needleman & Wunsch, J. Mol. Biol. 48:443 (1970), by the search for similarity method of Pearson & Lipman, Proc. Nat'l. Acad. Sci. USA 85:2444 (1988), by computerized implementations of these algorithms (GAP, BESTFIT, FASTA, and TFASTA in the Wisconsin Genetics Software Package, Genetics Computer Group, 575 Science Dr., Madison, Wis.), or by manual alignment and visual inspection (see, e.g., Current Protocols in Molecular Biology (Ausubel et al., eds. 1995 supplement)).

An exemplary algorithm that is suitable for determining percent sequence identity and sequence similarity are the BLAST and BLAST 2.0 algorithms, which are described in Altschul et al., Nuc. Acids Res. 25:3389-3402 (1977) and Altschul et al., J. Mol. Biol. 215:403-410 (1990), respectively. BLAST and BLAST 2.0 are used, with the parameters described herein, to determine percent sequence identity for the nucleic acids and proteins of the invention. Software for performing BLAST analyses is publicly available through the National Center for Biotechnology Information (http://www.ncbi.nlm.nih.gov/). This algorithm involves first identifying high scoring sequence pairs (HSPs) by identifying short words of length W in the query sequence, which either match or satisfy some positive-valued threshold score T when aligned with a word of the same length in a database sequence. T is referred to as the neighborhood word score threshold (Altschul et al., supra). These initial neighborhood word hits act as seeds for initiating searches to find longer HSPs containing them. The word hits are extended in both directions along each sequence for as far as the cumulative alignment score can be increased. Cumulative scores are calculated using, for nucleotide sequences, the parameters M (reward score for a pair of matching residues; always >0) and N (penalty score for mismatching residues; always <0). For amino acid sequences, a scoring matrix is used to calculate the cumulative score. Extension of the word hits in each direction are halted when: the cumulative alignment score falls off by the quantity X from its maximum achieved value; the cumulative score goes to zero or below, due to the accumulation of one or more negative-scoring residue alignments; or the end of either sequence is reached. The BLAST algorithm parameters W, T, and X determine the sensitivity and speed of the alignment. The BLASTN program (for nucleotide sequences) uses as defaults a wordlength (W) of 11, an expectation (E) of 10, M=5, N=−4 and a comparison of both strands. For amino acid sequences, the BLASTP program uses as defaults a wordlength of 3, and expectation (E) of 10, and the BLOSUM62 scoring matrix (see Henikoff & Henikoff, Proc. Natl. Acad. Sci. USA 89:10915 (1989)) alignments (B) of 50, expectation (E) of 10, M=5, N=−4, and a comparison of both strands.

The BLAST algorithm also performs a statistical analysis of the similarity between two sequences (see, e.g., Karlin & Altschul, Proc. Nat'l. Acad. Sci. USA 90:5873-5787 (1993)). One measure of similarity provided by the BLAST algorithm is the smallest sum probability (P(N)), which provides an indication of the probability by which a match between two nucleotide or amino acid sequences would occur by chance. For example, a nucleic acid is considered similar to a reference sequence if the smallest sum probability in a comparison of the test nucleic acid to the reference nucleic acid is less than about 0.2, more preferably less than about 0.01, and most preferably less than about 0.001.

An indication that two nucleic acid sequences or polypeptides are substantially identical is that the polypeptide encoded by the first nucleic acid is immunologically cross reactive with the antibodies raised against the polypeptide encoded by the second nucleic acid, as described below. Thus, a polypeptide is typically substantially identical to a second polypeptide, for example, where the two peptides differ only by conservative substitutions. Another indication that two nucleic acid sequences are substantially identical is that the two molecules or their complements hybridize to each other under stringent conditions, as described below. Yet another indication that two nucleic acid sequences are substantially identical is that the same primers can be used to amplify the sequence.

The phrase “selectively (or specifically) hybridizes to” refers to the binding, duplexing, or hybridizing of a molecule only to a particular nucleotide sequence under stringent hybridization conditions when that sequence is present in a complex mixture (e.g., total cellular or library DNA or RNA).

The phrase “stringent hybridization conditions” refers to conditions under which a probe will hybridize to its target subsequence, typically in a complex mixture of nucleic acids, but to no other sequences. Stringent conditions are sequence-dependent and will be different in different circumstances. Longer sequences hybridize specifically at higher temperatures. An extensive guide to the hybridization of nucleic acids is found in Tijssen, Techniques in Biochemistry and Molecular Biology—Hybridization with Nucleic Probes, “Overview of principles of hybridization and the strategy of nucleic acid assays” (1993). Generally, stringent conditions are selected to be about 5-10° C. lower than the thermal melting point (Tₘ) for the specific sequence at a defined ionic strength pH. The Tₘ is the temperature (under defined ionic strength, pH, and nucleic concentration) at which 50% of the probes complementary to the target hybridize to the target sequence at equilibrium (as the target sequences are present in excess, at Tₘ, 50% of the probes are occupied at equilibrium). Stringent conditions may also be achieved with the addition of destabilizing agents such as formamide. For selective or specific hybridization, a positive signal is at least two times background, preferably 10 times background hybridization. Exemplary stringent hybridization conditions can be as following: 50% formamide, 5×SSC, and 1% SDS, incubating at 42° C., or, 5×SSC, 1% SDS, incubating at 65° C., with wash in 0.2×SSC, and 0.1% SDS at 65° C.

Nucleic acids that do not hybridize to each other under stringent conditions are still substantially identical if the polypeptides which they encode are substantially identical. This occurs, for example, when a copy of a nucleic acid is created using the maximum codon degeneracy permitted by the genetic code. In such cases, the nucleic acids typically hybridize under moderately stringent hybridization conditions. Exemplary “moderately stringent hybridization conditions” include a hybridization in a buffer of 40% formamide, 1 M NaCl, 1% SDS at 37° C., and a wash in 1×SSC at 45° C. A positive hybridization is at least twice background. Those of ordinary skill will readily recognize that alternative hybridization and wash conditions can be utilized to provide conditions of similar stringency. Additional guidelines for determining hybridization parameters are provided in numerous reference, e.g., and Current Protocols in Molecular Biology, ed. Ausubel, et al.

For PCR, a temperature of about 36° C. is typical for low stringency amplification, although annealing temperatures may vary between about 32° C. and 48° C. depending on primer length. For high stringency PCR amplification, a temperature of about 62° C. is typical, although high stringency annealing temperatures can range from about 50° C. to about 65° C., depending on the primer length and specificity. Typical cycle conditions for both high and low stringency amplifications include a denaturation phase of 90° C.-95° C. for 30 sec-2 min., an annealing phase lasting 30 sec.-2 min., and an extension phase of about 72° C. for 1-2 min. Protocols and guidelines for low and high stringency amplification reactions are provided, e.g., in Innis et al. (1990) PCR Protocols, A Guide to Methods and Applications, Academic Press, Inc. N.Y.).

The terms “isolated,” “purified,” or “biologically pure” refer to material that is substantially or essentially free from components that normally accompany it as found in its native state. Purity and homogeneity are typically determined using analytical chemistry techniques such as polyacrylamide gel electrophoresis or high performance liquid chromatography. A protein that is the predominant species present in a preparation is substantially purified.

“Polypeptide” refers to a polymer in which the monomers are amino acids and are joined together through amide bonds, alternatively referred to as a “peptide.” The terms “peptide” and “polypeptide” encompass proteins. Unnatural amino acids, for example, β-alanine, phenylglycine and homoarginine are also included under this definition. Amino acids that are not gene-encoded may also be used in the present invention. Furthermore, amino acids that have been modified to include reactive groups may also be used in the invention. All of the amino acids used in the present invention may be either the D- or L-isomer. The L-isomers are generally preferred. In addition, other peptidomimetics are also useful in the present invention. For a general review, see, Spatola, A. F., in CHEMISTRY AND BIOCHEMISTRY OF AMINO ACIDS, PEPTIDES AND PROTEINS, B. Weinstein, eds., Marcel Dekker, New York, p. 267 (1983).

A “degraded post-translationally modified polypeptide” refers to the polypeptide fragments produced by site-specifically cleaving a post-translationally modified polypeptide at a site of post-translational modification using an endonuclease of the present invention.

The term “fragmentation pattern” refers to the configuration of the polypeptide fragments of the degraded post-translationally modified polypeptide as visualized or produced by an analytical method. A variety of analytical methods may be used to provide a fragmentation pattern. For example, where the analytical method is mass spectrometry, the fragmentation pattern is referred to as a “mass spectral fragmentation pattern.” Where the analytical method is two-dimensional electrophoresis, the fragmentation pattern is referred to as a “two-dimensional electrophoretic fragmentation pattern.”

The term “amino acid” refers to naturally occurring and synthetic amino acids, as well as amino acid analogs and amino acid mimetics that function in a manner similar to the naturally occurring amino acids. Naturally occurring amino acids are those encoded by the genetic code, as well as those amino acids that are later modified, e.g., hydroxyproline, γ-carboxyglutamate, and O-phosphoserine. Amino acid analogs refers to compounds that have the same basic chemical structure as a naturally occurring amino acid, i.e., an a carbon that is bound to a hydrogen, a carboxyl group, an amino group, and an R group, e.g., homoserine, norleucine, methionine sulfoxide, methionine methyl sulfonium. Such analogs have modified R groups (e.g., norleucine) or modified peptide backbones, but retain the same basic chemical structure as a naturally occurring amino acid. “Amino acid mimetics” refers to chemical compounds that have a structure that is different from the general chemical structure of an amino acid, but that functions in a manner similar to a naturally occurring amino acid.

“Solid support,” as used herein refers to a material that is substantially insoluble in a selected solvent system, or which can be readily separated (e.g., by precipitation) from a selected solvent system in which it is soluble. Solid supports useful in practicing the present invention can include groups that are activated or capable of activation to allow selected species to be bound to the solid support. A solid support can also be a substrate, for example, a chip, wafer or well, onto which an individual, or more than one compound, of the invention is bound.

By “host cell” is meant a cell that contains an expression vector and supports the replication or expression of the expression vector. Host cells may be prokaryotic cells such as E. coli, or eukaryotic cells such as yeast, insect, amphibian, or mammalian cells such as CHO, HeLa and the like, e.g., cultured cells, explants, and cells in vivo.

Introduction

One surprise of the human genome sequence was that there are far fewer genes than many had predicted. Instead, much of the complexity of higher organisms is predicted to reside in the specific modification of proteins, and piecing together this extraordinarily complex web of post-translational modifications is one of the great remaining frontiers in biology. For example, phosphorylation is the most ubiquitous and important of these modifications (one-third of all cellular proteins contain covalently bound phosphate), and understanding the molecular logic of protein phosphorylation will be a major step toward decoding biological processes. New tools that will aid in the understanding of post-translational modifications on a genome wide scale are needed. In view of the importance of phosphorylation, the present invention is illustrated by reference to ascertaining the phosphorylation pattern of a peptide. The focus on phosphorylation is for clarity of illustration and does not limit the scope of the invention.

Mapping the Sites of Polypeptide Post-Translational Modifications

In a first aspect, the invention provides a method of mapping a site of polypeptide post-translational modifications. The method includes site-specifically cleaving a peptide bond of the post-translationally modified polypeptide with an endopeptidase at a site of post-translational modification to produce a degraded post-translationally modified polypeptide. After cleavage at the site of post-translational modification, the site of post-translational modification is determined.

Site-specific cleavage refers to peptide bond hydrolysis at a preferred site in a polypeptide. For example, many endopeptidases cleave the amide backbone of polypeptides site-specifically at a preferred amino acid residue and/or residues. Endopeptidases that site-specifically cleave polypeptides include, for example, chymotrypsin, which site-specifically cleaves at phenylalanine, tryptophan and tyrosine residues; trypsin, which exhibits preferential cleavage at lysine and arginine residues; elastase, which site-specifically cleaves at alanine residues, and subtilisin, which site-specifically cleaves at tyrosine and phenylalanine residues. Similarly, endopeptidases of the present invention that cleave site-specifically at a site of post-translational modification exhibit preferential cleavage at amino acid residues that have been post-translationally modified. More detailed information regarding known protease cleavage sites may be found, for example, in Matayoshi et al. Science 247: 954 (1990); Dunn et al. Meth. Enzymol. 241: 254 (1994); Seidah et al. Meth. Enzymol. 244: 175 (1994); Thornberry, Meth. Enzymol. 244: 615 (1994); Weber et al. Meth. Enzymol. 244: 595 (1994); Smith et al. Meth. Enzymol. 244: 412 (1994); Bouvier et al. Meth. Enzymol. 248: 614 (1995), and Hardy et al., in AMYLOID PROTEIN PRECURSOR IN DEVELOPMENT, AGING, AND ALZHEIMER'S DISEASE, ed. Masters et al. pp. 190-198 (1994).

A wide variety of methods are useful in determining the specificity of site-specific cleavage. For example, a test polypeptide containing a fluorescent donor-fluorescent quencher pair can be used to measure the kinetics of cleavage by an endopeptidase. See, for example, Meldal et al., Anal. Biochem. 195:141-7(1991) and Examples section. The cleavage kinetics of a test polypeptide containing a particular post-translational modification may be measured and subsequently compared to the cleavage kinetics of a series of control polypeptides that do not contain the post-translational modification. Typically, the test polypeptide contains the same amino acid sequence as the control peptides, with the exception that the amino acid containing the post-translational modification in the test polypeptide is substituted for another amino acid in the control polypeptide amino acid sequences. The amino acid containing the post-translational modification may be substituted, for example, with an unmodified natural amino acid, an unmodified non-natural amino acid, the same amino acid containing a different post-translational modification, a different amino acid containing the same post-translational modification, and/or a different amino acid containing a different post-translational modification.

In an exemplary embodiment, an endopeptidase site-specifically cleaves a polypeptide at a site of post-translational modification when the k_{cat}/Kₘ ratio for the post-translationally modified test polypeptide is higher than the k_{cat}/Kₘ ratio for a control polypeptide or a series of control peptides that do not contain the post-translational modification. In another exemplary embodiment, an endopeptidase site-specifically cleaves at a site of post-translational modification when the k_{cat}/Kₘ ratio is at least about 1.1, 1.2, 1.3, 1.4, 1.5, 1.6, 1.7, 1.8, or 1.9 times higher for the modified test polypeptide than the k_{cat}/Kₘ ratio for the control polypeptide(s). In another exemplary embodiment, an endopeptidase site-specifically cleaves at a site of post-translational modification when the k_{cat}/Kₘ ratio is at least about 2, 3, 4, 5, 6, 7, 8, 9, or 10 fold higher for the modified test polypeptide than the k_{cat}/Kₘ ratio for the control polypeptide(s).

The endopeptidases of the present invention are capable of site-specifically cleaving a polypeptide at a site containing any suitable post-translational modification. Over 300 post-translational modifications are currently known. See the world wide web at URL http://www.abrf.org/index.cfin/dm.home?AvgMass=all, Delta Mass, A Database of protein Post-Translational Modifications. Exemplary suitable post-translational modifications include phosphorylation, sulfonation, glycosylation, acetylation, methylations, ADP-ribosylation, methionine oxidation, cysteine oxidation, cysteine lipidation, farnesylation, and geranylation.

In an exemplary embodiment, the post-translational modification is phosphorylation. Typically, post-translational phosphorylation occurs at a tyrosine, serine, and or threonine. In a related exemplary embodiment, the endopeptidase site-specifically cleaves a polypeptide at phosphorylated tyrosine, serine, or threonine. In another related embodiment, the endopeptidase site-specifically cleaves a polypeptide at a phosphorylated tyrosine.

In another exemplary embodiment, the post-translational modification is sulfonation. In a related embodiment, the endopeptidase site-specifically cleaves a polypeptide at a sulfonated tyrosine.

The present method includes site-specifically cleaving a post-translationally modified polypeptide at a site of post-translational modification with an endopeptidase. Typically, an endopeptidase that cleaves at a site of post-translational modification hydrolyzes a peptide bond between two adjacent amino acid residues, wherein the peptide bond is within 10 amino acids in either direction of the polypeptide amino acid containing the post-translational modification. For example, where a tyrosine is phosphorylated, the endopeptidases of the present invention will site-specifically cleave the polypeptide at a peptide bond within 10 amino acid residues, in either the N-terminal direction or the C-terminal direction, of the phosphorylated tyrosine. Thus, site-specific cleavage at a site of post translational modification typically refers to cleavage at a peptide bond between two amino acids, wherein the peptide bond is within ten amino acids in either direction of the post-translationally modified amino acid.

In an exemplary embodiment, the endopeptidase site-specifically cleaves a post-translationally modified peptide at a peptide bond within 1, 2, 3, 4, 5, 6, 7, 8, 9, or 10 amino acids of the post-translationally modified amino acid. In another exemplary embodiment, the endopeptidase site-specifically cleaves a post-translationally modified peptide at a peptide bond between the post-translationally modified amino acid and the amino acid immediately C-terminal to the post-translationally modified peptide or the amino acid immediately N-terminal to the post-translationally modified peptide. Thus, the site of cleavage may be at the peptide bond between the post-translationally modified amino acid and an amino acid adjacent to the post-translationally modified amino acid.

The present method also includes determining the site of post-translational modification after cleavage at the site of post-translational modification using the endopeptidases of the present invention. A variety of methods are useful in determining the site of post-translational modification after cleavage. Typically, the methods involve analyzing the degraded post-translationally modified polypeptide produced by cleaving the post-translationally modified polypeptide with an endopeptidase of the present invention. Exemplary methods include determining the fragmentation pattern of the polypeptide fragments and comparing the pattern to a known or predicted pattern, determining the size of the polypeptide fragments, determining the sequence of the polypeptide fragments produced, and quantitating the amount of polypeptide fragments produced. A variety of analytical tools may be employed in conjunction with these methods, including, gel electrophoresis (such as single and multi-dimensional electrophoresis), mass spectrometry (including mass spectrometry polypeptide sequencing techniques), high performance liquid chromatography (HPLC), nuclear magnetic resonance (NMR), capillary gel electrophoresis, affinity chromatography, Edman degradation, high throughput protein chip technology, and the like.

In an exemplary embodiment, the site of post-translational modification is determined by sequencing the polypeptide fragments produced by cleaving the polypeptide with the endopeptidases of the present invention. Sequencing can be accomplished using any suitable technique, such as Edman degradation or mass spectrometry.

In another exemplary embodiment, the site of post-translational modification is determined from the fragmentation pattern of the degraded post-translationally modified polypeptide produced by the endopeptidases of the current invention. The fragmentation pattern may be compared to predicted fragmentation patterns of known polypeptide sequences, thereby identifying the sites of post-translational modifications. Alternatively, the fragmentation pattern may be compared to a plurality of empirically produced fragmentation patterns to determine the site of post-translational modification. After cleavage, fragmentation patterns may be produced by a variety of methods, including, for example, mass spectrometry and two dimensional gel electrophoresis. These and other methods are discussed in more detail in the “Informatics” section below.

Post-translationally modified polypeptides of use in the present invention may be of any biological or synthetic origin. For example, the post-translationally modified polypeptide may be produced using known chemical techniques, such as solid phase peptide synthesis on a solid support, wherein post-translationally modified amino acids (either protected or unprotected) are incorporated into the polypeptide chain during synthesis (see Stewart et al., Solid Phase Peptide Synthesis, Second Edition (1984)). Alternatively, an unmodified polypeptide chain may be chemically synthesized and subsequently contacted with an enzyme in vitro to create a synthetic post-translationally modified polypeptide. In an exemplary embodiment, an unmodified polypeptide is synthesized using solid phase peptide synthesis and subsequently phosphorylated with a protein tyrosine kinase to produce a post-translationally modified polypeptide.

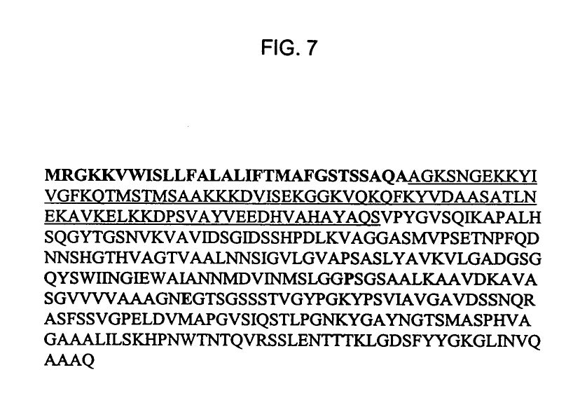

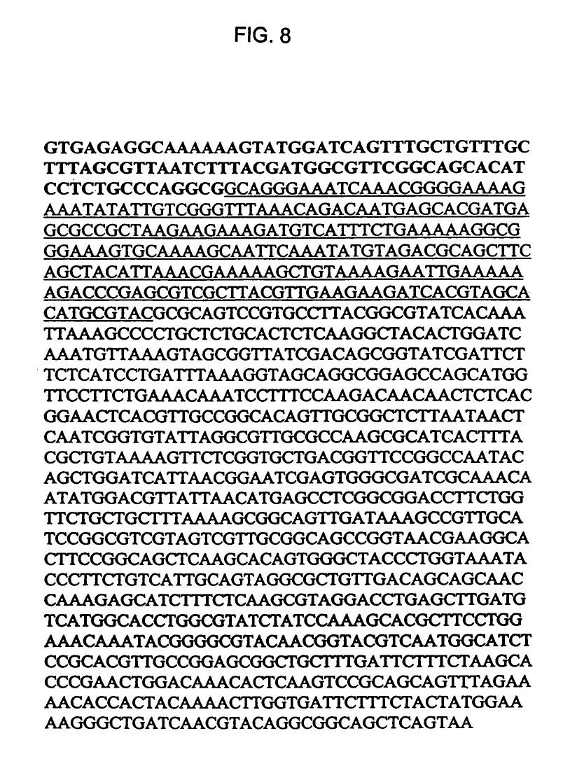

In another exemplary embodiment, the post-translationally modified polypeptide is produced in a cell. Using recombinant methods, a secretory signal sequence may be included in the polypeptide sequence so that the post-translationally modified polypeptide is secreted from the cell, thus simplifying purification procedures. Exemplary amino acid signal sequences and nucleic acid sequences that encode the signal sequence are described, for example, in Wells et al., Nucleic Acids Research, 11:7911-7925 (1983), and in FIGS. 7 and 8 (in bold). In another exemplary embodiment, recombinant methods may be used to include an endopeptidase prodomain, such as the subtilisin prodomain shown in FIGS. 7 and 8 (underlined).

In another exemplary embodiment, the post-translationally modified polypeptide is produced by a diseased host, wherein at least one post-translational modification is a marker of disease. In a related embodiment, the post-translational modification that is a disease marker is sulfonation of a tyrosine. In another exemplary embodiment, the post-translationally modified polypeptide is derived from a non-diseased host. In another exemplary embodiment, the post-translationally modified polypeptide is targeted for cleavage with endopeptidases of the present invention by at least partially purifying the post-translationally modified polypeptide before cleavage with the endopeptidase.

Endopeptidases

In another aspect, the present invention provides an endopeptidase that site-specifically cleaves a peptide bond of a post-translationally modified polypeptide at a site of post-translational modification, wherein the endopeptidase comprises an active site that binds to the site of post-translational modification.

The active site of an endopeptidase of the present invention refers to the area of the endopeptidase that binds to the post-translationally modified polypeptide and contains the amino acids side chains involved in peptide bond hydrolysis. Typically, the active site contains amino acids that bind to the post-translational modification itself, in addition to other areas of the post-translationally modified polypeptide, such as other amino acid side chains or polypeptide backbone carbonyl and/or amine groups. The binding and catalytic properties of an active site is determined by the three dimensional arrangement of the amino acid side chains within the active site.

The active site of the endopeptidase may bind to the post-translational modification using any suitable molecular binding interaction. Typically, the binding interaction is a non-covalent interaction. Useful non-covalent binding interactions include, for example, ionic interactions, hydrogen bonding, Van der Waals interactions, dipole-dipole interactions, pi-pi stacking interactions, and/or hydrophobic interactions. The active site may also increase binding interactions to the post-translational modification by containing a suitable space for the post-translational modification to fit within the active site, thus avoiding steric clashes between the endopeptidase active site amino acids and the post-translational modification.

A variety of post-translational modifications are bound by the endopeptidases of the present invention. For example, where the post-translational modification is phosphorylation, the endopeptidase active site typically contains one or more positively charged amino acid side chains that ionically bind to the negatively charged phosphate moiety. In another example, where the post-translational modification is glycosylation, the endopeptidase active site comprises cyclic amino acid side chain residues that stack above or below a sugar ring of the glycosylation modification.

Endopeptidases are proteases that cleave a non-terminal peptide bond of a polypeptide substrate. Proteases have been found to contain common structural features (see Stawiski et al., Proc. Natl. Acad. Sci., 97: 3954-3958 (2000)). For example, relative to proteins of similar size, proteases have smaller than average surface areas, smaller radii of gyration, higher Cα densities, are more tightly packed than other proteins, and have fewer helices and more loops. Based on these structural similarities, protease function has been predicted with over 86% accuracy from the primary amino acid sequence of polypeptides (Id.).

In an exemplary embodiment, the endopeptidase is a serine protease. Serine proteases of the present invention differ from previously known serine proteases in that they are able to site-specifically cleave a post-translationally modified polypeptide at a site of post-translational modification. However, the serine proteases of the present invention typically retain the features of the enzymes within the sub-subclass EC 3.4.21. In addition, the serine proteases of the present invention retain the “catalytic triad” active site structural motif common to all previously known serine proteases, as explained below.

Endopeptidases within the serine protease family are structurally related through a common active site structural motif (see Stroud, Sci. Am., 231: 74-88 (1974)). The active site structural motif is commonly referred to as the “catalytic triad,” which includes a specific three-dimensional arrangement of three amino acids: serine, histidine, and aspartate (see Rusell, J. Mol. Biol., 279: 1211-1227 (1998)). The three amino acids act in concert to cleave the peptide bond of a polypeptide. The catalytic mechanism involves attack of the serine hydroxyl side chain onto the carbonyl moiety of the peptide bond to form a tetrahedral intermediate, followed by general acid catalysis of the intermediate by the aspartate-polarized histidine (see Voet et al., Biochemistry, Second Ed., p. 395 (1995)).

The three-dimensional structure of the catalytic triad is sufficiently similar between the members of the serine protease family that the serine protease catalytic triad can be accurately detected from the amino acid sequence alone (see Fischer et al., Protein Sci. 3: 769-788 (1994); Wallace et al., Protein Sci., 5: 1001-1013 (1996); Wallace et al., Protein Sci., 6: 2308-2323 (1997); Rusell, J. Mol. Biol., 279: 1211-1227 (1998)). Methods for determining the presence of the serine protease catalytic triad typically involve predicting the angles and distances between amino acids in the active site of a protein using computer-based algorithms that analyze the primary structure of the protein. In some methods, the amino acid sequence is additionally considered in determining serine protease identity (see Rusell, J. Mol. Biol., 279: 1211-1227 (1998)). Although all serine proteases may not share a high degree of amino acid sequence identity, one skilled in the art will recognize common serine protease structures by analyzing the three dimensional structure of the active site and detecting the presence of the serine/histidine/aspartate catalytic triad. In fact, the three dimensional spatial relationships of the active site of enzymes are often more informative than the one-dimensional primary sequence alone (Rusell, J. Mol. Biol., 279: 1211-1227 (1998)). For example, although trypsin, chymotrypsin and elastase share similar function, three dimensional backbone structure, and catalytic triad structure, only 24 percent of the amino acids are common to all three of these enzymes (see Stroud, Sci. Am., 231: 74-88 (1974)).

In another related embodiment, the endopeptidase is a trypsin serine protease. Trypsin serine proteases of the present invention differ from previously known trypsin serine proteases in that they are able to site-specifically cleave a post-translationally modified polypeptide at a site of post-translational modification. However, the trypsin serine proteases of the present invention retain the three dimensional catalytic triad and the non-active site elements of secondary and tertiary structure of previously known trypsin serine proteases. In an exemplary embodiment, trypsin serine proteases are those having the serine protease catalytic triad structure and the following structural characteristics according to the CATH protein structural classification: class 2 (mainly beta), architecture 2.40 (barrel), topology 2.40.10 (thrombin subunit H), homologous superfamily 2.40.10.10 (trypsin-like serine protease), and sequence family 2.40.10.10.2 (trypsin-like serine protease). The trypsin serine proteases of the present invention typically retain the three dimensional catalytic triad and the non-active site elements of secondary and tertiary structure of those enzymes included within sub-subclass EC 3.4.21.4.

In another related embodiment, the endopeptidase is a subtilisin. Subtilisins of the present invention differ from previously known subtilisins in that they are able to site-specifically cleave a post-translationally modified polypeptide at a site of post-translational modification. However, the subtilisins of the present invention retain the three dimensional catalytic triad and the non-active site elements of secondary and tertiary structure of known subtilisin enzymes. In an exemplary embodiment, the subtilisin of the present invention is a single polypeptide chain that folds into three distinct regions and contains eight α-helices (designated A-H, see Wright et al., Nature, 221: 235-242 (1969)). In another exemplary embodiment, the subtilisin of the present invention retains the three dimensional catalytic triad and the non-active site elements of secondary and tertiary structure of those enzymes included within sub-subclass EC 3.4.21.62.

In another exemplary embodiment, the endopeptidase is a cysteine protease. Common active site structural motifs have been used to successfully identify members of the cysteine protease family (see Rusell, J. Mol. Biol., 279: 1211-1227 (1998)). Although cysteine proteases lack the serine/histidine/aspartate catalytic triad of the serine protease family, similarity in the overall tertiary side chain pattern and shape of the active site may be used to identify members of the cysteine protease family. In a related exemplary embodiment, the cysteine protease is any enzyme of the sub-subclass EC 3.4.22, which consists of proteinases characterized by having a cysteine residue at the active site and by being irreversibly inhibited by sulfhydryl reagents such as iodoacetate. Mechanistically, in catalyzing the cleavage of a peptide amide bond, cysteine proteases form a covalent intermediate, called an acyl enzyme, that involves a cysteine and a histidine residue in the active site (Cys25 and His159 according to papain numbering, for example).

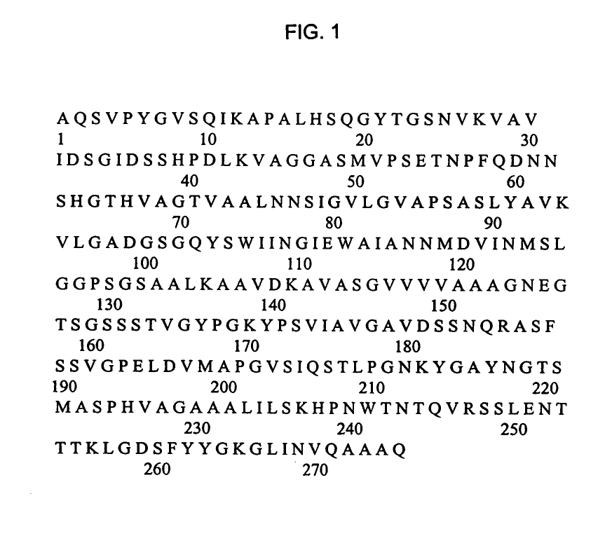

In another exemplary embodiment, the endopeptidase of the present invention is encoded by a nucleic acid sequence that hybridizes under highly stringent hybridization conditions to a nucleic acid encoding a polypeptide comprising an amino acid sequence of FIG. 1. In a related embodiment, the amino acid sequence additionally contains a prodomain sequence as shown in FIG. 7 (underlined). In another related embodiment, the amino acid sequence additionally contains a signal sequence as shown in FIG. 7 (in bold). Typically, the hybridization reaction is incubated at 42° C. in a solution comprising 50% formamide, 5×SSC and 1% SDS, and washed at 65° C. in a solution comprising 0.2×SSC and 0.1% SDS. In a related embodiment, the endopeptidase contains at least one amino acid substitution selected from P129G, E156R, S191K, G166K, G127S, E156K, P129K, P129R, S159R, and E156G (see FIG. 1). In another related embodiment, the endopeptidase contains one of the following combinations of substitution point mutations: G127S and E156R; P129G and E156R; P129G and E156K; E156R and S191K; P129K and E156R; P129R and E156K; E156K and G166K; E156K and S191K; E156K and S191K and S159R; P129R and E156R; and E156G and G166K. In another related embodiment, the endopeptidase contains a subsequence as described above and contains one or two amino acid substitutions selected from P129G, E156R, S191K, G166K, and G127S.

In another exemplary embodiment, the endopeptidase contains a subsequence having at least 70% amino acid sequence identity to an amino acid sequence of FIG. 1. In a related embodiment, the subsequence has 75%, 76%, 77%, 78%, 79%, 80%, 85%. 86%, 87%, 88%, 89%, 90%, 91%, 92%, 93%, 94%, 95%, 96%, 97%, or 98% amino acid sequence identity.

In another related embodiment, the endopeptidase contains a subsequence as described above and contains at least one amino acid substitution selected from P129G, E156R, S191K, G166K, G127S, E156K, P129K, P129R, S159R, and E156G. In another related embodiment, the endopeptidase contains a subsequence as described above and contains one of the following combinations of substitution point mutations: G127S and E156R; P129G and E156R; P129G and E156K; E156R and S191K; P129K and E156R; P129R and E156K; E156K and G166K; E156K and S191K; E156K and S191K and S159R; P129R and E156R; and E156G and G166K. In another related embodiment, the endopeptidase contains a subsequence as described above and contains one or two amino acid substitutions selected from P129G, E156R, S191K, G166K, and G127S. In another related embodiment, the amino acid sequence additionally contains a prodomain sequence as shown in FIG. 7 (underlined). In another related embodiment, the amino acid sequence additionally contains a signal sequence as shown in FIG. 7 (in bold).

In another exemplary embodiment, the endopeptidase of the present invention is encoded by an expression vector. In another exemplary embodiment, a host cell contains the expression vector. A variety of host cells may be used in the methods of the present invention (see “Expression in Eukaryotes and Prokaryotes” below). In an exemplary embodiment, the host cell is B. subtilis.

Production of Endopeptidases

In another aspect, the endopeptidases of the present invention are produced by a method that includes introducing one or more point mutations into a model endopeptidase at one or more candidate amino acid positions in an active site of the model endopeptidase to produce a plurality of candidate endopeptidases. At least one of the plurality of the candidate endopeptidases is an endopeptidase of the present invention that site-specifically cleaves a peptide bond of a post-translationally modified polypeptide at a site of post-translational modification. The endopeptidase that site-specifically cleaves at the site of post-translational modification is then identified. Typically, the endopeptidase identification is accomplished by assaying the candidate endopeptidases.

A variety of model endopeptidases are useful in the current invention. Typically, the model endopeptidase of the current invention cleaves a peptide bond of a polypeptide at a specific site, such as chymotrypsin, which site-specifically cleaves at phenylalanine, tryptophan and tyrosine residues; trypsin, which exhibits preferential cleavage at lysine and arginine residues; and elastase, which site-specifically cleaves at alanine residues. Exemplary model endopeptidases include, for example, serine proteases within the sub-subclass EC 3.4.21 and cysteine proteases within the sub-subclass cysteine 3.4.22. In an exemplary embodiment, the serine protease is a trypsin endopeptidase within the sub-subclass EC 3.4.21.4 or a subtilisin endopeptidase within the sub-subclass EC 3.4.21.62.

In an exemplary embodiment, the model endopeptidase is encoded by a nucleic acid sequence that hybridizes under highly stringent hybridization conditions to a nucleic acid encoding a polypeptide comprising an amino acid sequence of FIG. 1, wherein the hybridization reaction is incubated at 42° C. in a solution comprising 50% formamide, 5×SSC and 1% SDS, and washed at 65° C. in a solution comprising 0.2×SSC and 0.1% SDS. In a related embodiment, the amino acid sequence additionally contains a prodomain sequence as shown in FIG. 7 (underlined). In another related embodiment, the amino acid sequence additionally contains a signal sequence as shown in FIG. 7 (in bold).

In another related embodiment, model endopeptidase contains a subsequence having at least 70% amino acid sequence identity to an amino acid sequence of FIG. 1. In another related embodiment, the subsequence has at least 75%, 76%, 77%, 78%, 79%, 80%, 85%. 86%, 87%, 88%, 89%, 90%, 91%, 92%, 93%, 94%, 95%, 96%, 97%, 98%, 995, or 100% sequence identity to an amino acid sequence of FIG. 1. In a related embodiment, the amino acid sequence additionally contains a prodomain sequence as shown in FIG. 7 (underlined). In another related embodiment, the amino acid sequence additionally contains a signal sequence as shown in FIG. 7 (in bold).

Endopeptidases of the present invention are typically produced by a method that includes introducing one or more point mutations into the active site of a model endopeptidase. As explained above, a point mutation refers to a deletion, addition, or substitution at a designed amino acid position in an amino acid or nucleotide sequence. In an exemplary embodiment, the one or more point mutations is one or more amino acid substitutions. In another exemplary embodiment, one or two substitution point mutations are introduced into the active site of a model endopeptidase.

The point mutations are introduced at candidate amino acid positions within the active site of the model endopeptidase preferably after examining the three dimensional structure of the model endopeptidase. In an exemplary embodiment, before introducing one or more point mutations to a model endopeptidase at one or more candidate amino acid positions, the one or more candidate amino acid positions are identified by a method that includes generating a three-dimensional structure of the model endopeptidase active site.

In another exemplary embodiment, the one or more candidate amino acid positions are identified by a method that includes generating a three-dimensional structure of the model endopeptidase active site and a three-dimensional structure of the post-translationally modified polypeptide. The three-dimensional structure of the model endopeptidase active site is compared with the site of the post-translationally modified polypeptide, thereby identifying one or more candidate amino acid positions. Point mutations are then introduced into the candidate amino acid positions to generate a plurality of candidate endopeptidases. Upon introduction of one or more point mutations at one or more of the candidate amino acid positions, a plurality of candidate endopeptidases is produced. Typically, at least one of the plurality of candidate endopeptidases is an endopeptidase that site-specifically cleaves a peptide bond of a post-translationally modified polypeptide at a site of post-translational modification.

In a related embodiment, amino acid substitutions at the candidate amino acid positions is rationally designed by generating a three-dimensional structure of potential candidate endopeptidases before generating the actual candidate endopeptidases using recombinant techniques. The three-dimensional structure of potential candidate endopeptidase is compared to the post-translationally modified polypeptide to determine whether the point mutation provides one or more binding interactions with the post-translationally modified polypeptide.

For example, where the post-translationally modified polypeptide is a phosphotyrosine polypeptide, the three-dimensional structure of the phosphotyrosine polypeptide is compared to the three-dimensional structure of the model endopeptidase, such as trypsin. A candidate amino acid position is identified in the trypsin active site that is potentially within ionic bonding distance of the phosphate moiety of the phosphotyrosine polypeptide. However, the amino acid that occupies the candidate amino acid position (for example, an alanine or valine residue) in trypsin will typically not be capable of forming an ionic bond with the negatively charged phosphate moiety. Therefore, a three dimensional structure of a potential candidate endopeptidase is generated that contains, for example, an arginine substitution at the candidate amino acid position. The structure of the potential candidate endopeptidase is then compared with the phosphotyrosine structure to determine whether or not the arginine forms an ionic bond with the phosphate moiety. If a bond appears to be possible from the comparison, a candidate endopeptidase is generated containing an arginine substitution point mutation. The candidate endopeptidase is then assayed to determine whether or not it site-specifically cleaves the phosphotyrosine at the site of phosphorylation.

The amino acid substitution will typically depend on the type of interaction desired. For example, where the post-translationally modified polypeptide contains a charged moiety, an ionic interaction may be desired. Amino acids with side chains containing a charged side chain may be substituted for the amino acid in the model peptide at a candidate amino acid position within ionic bonding distance of the charged moiety. Amino acids with side chains capable of forming an ionic bond with a negatively charged moiety include lysine (PK 10.54), arginine (pK 12.48) and histidine (pK 6.04). Amino acids with side chains capable of forming an ionic bond with a positively charged moiety include aspartic acid (pK 3.9), glutamic acid (pK 4.07), tyrosine (pK 10.46), and cysteine (pK 8.37). Amino acids with side chains capable of forming a pi-pi stacking interaction with a polypeptide aromatic moiety include phenylalanine, tryptophan, and tyrosine. Amino acids with side chains capable of forming hydrogen bonds with a polypeptide moiety include methionine, tryptophan, serine, threonine, asparagine, glutamine, tyrosine. cysteine, lysine, arginine, histidine, aspartic acid and glutamic acids. Amino acids with small side chains capable of avoiding steric clashes include glycine, alanine and valine. Amino acids with side chains capable of participating in hydrophobic interactions include alanine, valine, leucine, isoleucine, phenylalanine and proline. In an exemplary embodiment, where the post-translationally modified peptide contains a charged moiety, only a single charged amino acid is introduced in the active site of the model endopeptidase. In a related embodiment, only a single positively charged amino acid is introduced into the active site of a model endopeptidase to bind to a phosphorylated polypeptide.

Typically, a computer program is used to generate the three-dimensional structures of the post-translationally modified polypeptide, the model endopeptidase, and/or the potential candidate endopeptidases. The three-dimensional computer-generated structures may be based on X-ray crystallographic data or NMR data. Alternatively, the structures may be predicted from the primary structure of the endopeptidase and/or post-translationally modified peptide using a computer algorithm.

The model endopeptidase structure may be compared with the post-translationally modified polypeptide structure to identify candidate amino acid positions. In addition, the potential candidate endopeptidase structures may be compared with the post-translationally modified polypeptide structure to identify amino acid substitutions suitable for of binding the post-translationally modified polypeptide. A variety of methods are useful in comparing the three-dimensional structures of the model endopeptidase and/or potential candidate endopeptidases with the post-translationally modified polypeptide. The comparison typically includes the use of a computer-based algorithm that identifies binding interactions, potential binding interactions, and/or steric clashes between the post-translationally modified polypeptide and the amino acid side chains and peptide backbone of the model endopeptidase or potential candidate endopeptidase active site. Amino acid side chains that sterically clash with or surround the post translationally modified polypeptide are typically identified as candidate amino acid positions.

A variety of useful computer based algorithms are useful in the present invention. Useful programs include, for example, InsightII (Accelrys), 3D-Dock (Imperial College), HEX (Aberdeen University), DOT (UCSD), ICM and input scripts for docking (Scripps), GRAMM (SUNY/MUSC), PPD (Colombia), BIGGER (Universidade Nova Lisboa), VAJDA/Camacho refinement (University of Boston), DOCK 4.0 (UCSF), Autodock (Scripps), FlexX(GMD-SCAI, BioSolvIT GmbH), Darwin (University of Pennsylvannia), and ZDOCK (University of Boston).

A plurality of candidate endopeptidases are produced by introducing point mutations at each of the candidate amino acid positions. The candidate endopeptidases may contain one point mutation or a combination of point mutations at the candidate amino acid positions. To identify endopeptidases that site-specifically cleave a peptide bond of a post-translationally modified polypeptide at a site of post-translational modification, the candidate endopeptidases are typically tested in a cleavage assay.

A variety of cleavage assays are useful in the current invention. Typically, the assay involves contacting a candidate endopeptidase with a test polypeptide comprising a post-translational modification. After contacting the test polypeptide with the candidate endopeptidase, the test polypeptide or test polypeptide fragments are analyzed to determine whether or not the candidate endopeptidase site-specifically cleaved the peptide bond of the test polypeptide at the site of post-translational modification. Methods of analyzing the test polypeptide or fragments thereof include, for example, sequencing methods (such as Edman degradation and Mass spectrometry), NMR, gel electrophoresis, capillary gel electrophoresis, HPLC, colorimetric assays, and the like. In an exemplary embodiment, the test peptide contains a fluorescent donor-fluorescent quencher pair, as described above.

In an exemplary embodiment, the methods of producing the endopeptidases of the present invention further includes, after performing the cleavage assays, producing one or more additional candidate endopeptidases. The one or more additional candidate endopeptidases are typically produced by introducing a new point mutation or new combination of point mutations in the active site of a candidate endopeptidase to optimize cleavage specificity. The candidate amino acid sites and the identity of the amino acid substitution is typically based on the results of the cleavage assay. The one or more additional candidate endopeptidases are then tested in a second set of cleavage assay. Thus, the methods of the present invention also include an iterative design process, in which the steps described herein are repeated to produce an optimized endopeptidase that site-specifically cleaves a post-translationally modified polypeptide.

General Recombinant DNA Methods

The production of endopeptidases of the current invention relies on routine techniques in the field of recombinant genetics. Basic texts disclosing the general methods of use in this invention include Sambrook et al., Molecular Cloning, A Laboratory Manual (2nd ed. 1989); Kriegler, Gene Transfer and Expression: A Laboratory Manual (1990); and Current Protocols in Molecular Biology (Ausubel et al., eds., 1994)).

For nucleic acids, sizes are given in either kilobases (Kb) or base pairs (bp). These are estimates derived from agarose or acrylamide gel electrophoresis, from sequenced nucleic acids, or from published DNA sequences. For proteins, sizes are given in kilodaltons (kD) or amino acid residue numbers. Protein sizes are estimated from gel electrophoresis, from sequenced proteins, from derived amino acid sequences, or from published protein sequences.

Oligonucleotides that are not commercially available can be chemically synthesized according to the solid phase phosphoramidite triester method first described by Beaucage & Caruthers, Tetrahedron Letts. 22:1859-1862 (1981), using an automated synthesizer, as described in Van Devanter et. al., Nucleic Acids Res. 12:6159-6168 (1984). Purification of oligonucleotides is by either native acrylamide gel electrophoresis or by anion-exchange HPLC as described in Pearson & Reanier, J. Chrom. 255:137-149 (1983).

The sequence of the cloned genes and synthetic oligonucleotides can be verified after cloning using, e.g., the chain termination method for sequencing double-stranded templates of Wallace et al., Gene 16:21-26 (1981).

Expression in Prokaryotes and Eukaryotes

To obtain high level expression of a cloned gene, such as those cDNAs encoding endopeptidases, one typically subclones endopeptidase into an expression vector that contains a strong promoter to direct transcription, a transcription/translation terminator, and if for a nucleic acid encoding a protein, a ribosome binding site for translational initiation. Suitable bacterial promoters are well known in the art and described, e.g., in Sambrook et al., and Ausubel et al, supra. Bacterial expression systems for expressing endopeptidases are available in, e.g., E. coli, Bacillus sp., and Salmonella (Palva et al., Gene 22:229-235 (1983); Mosbach et al., Nature 302:543-545 (1983). Kits for such expression systems are commercially available. Eukaryotic expression systems for mammalian cells, yeast, and insect cells are well known in the art and are also commercially available.

Selection of the promoter used to direct expression of a heterologous nucleic acid depends on the particular application. The promoter is preferably positioned about the same distance from the heterologous transcription start site as it is from the transcription start site in its natural setting. As is known in the art, however, some variation in this distance can be accommodated without loss of promoter function.

In addition to the promoter, the expression vector typically contains a transcription unit or expression cassette that contains all the additional elements required for the expression of the endopeptidase encoding nucleic acid in host cells. A typical expression cassette thus contains a promoter operably linked to the nucleic acid sequence encoding endopeptidase and signals required for efficient polyadenylation of the transcript, ribosome binding sites, and translation termination. Additional elements of the cassette may include enhancers and, if genomic DNA is used as the structural gene, introns with functional splice donor and acceptor sites.

In addition to a promoter sequence, the expression cassette should also contain a transcription termination region downstream of the structural gene to provide for efficient termination. The termination region may be obtained from the same gene as the promoter sequence or may be obtained from different genes.

The particular expression vector used to transport the genetic information into the cell is not particularly critical. Any of the conventional vectors used for expression in eukaryotic or prokaryotic cells may be used. Standard bacterial expression vectors include plasmids such as pBR322 based plasmids, pSKF, pET23D, and fusion expression systems such as MBP, GST, and LacZ. Epitope tags can also be added to recombinant proteins to provide convenient methods of isolation, e.g., c-myc.

Expression vectors containing regulatory elements from eukaryotic viruses are typically used in eukaryotic expression vectors, e.g., SV40 vectors, papilloma virus vectors, and vectors derived from Epstein-Barr virus. Other exemplary eukaryotic vectors include pMSG, pAV009/A⁺, pMTO10/A⁺, pMAMneo-5, baculovirus pDSVE, and any other vector allowing expression of proteins under the direction of the CMV promoter, SV40 early promoter, SV40 later promoter, metallothionein promoter, murine mammary tumor virus promoter, Rous sarcoma virus promoter, polyhedrin promoter, or other promoters shown effective for expression in eukaryotic cells.

Expression of proteins from eukaryotic vectors can be also be regulated using inducible promoters. With inducible promoters, expression levels are tied to the concentration of inducing agents, such as tetracycline or ecdysone, by the incorporation of response elements for these agents into the promoter. Generally, high level expression is obtained from inducible promoters only in the presence of the inducing agent; basal expression levels are minimal. Inducible expression vectors are often chosen if expression of the protein of interest is detrimental to eukaryotic cells.

Some expression systems have markers that provide gene amplification such as thymidine kinase and dihydrofolate reductase. Alternatively, high yield expression systems not involving gene amplification are also suitable, such as using a baculovirus vector in insect cells, with endopeptidase encoding sequence under the direction of the polyhedrin promoter or other strong baculovirus promoters.

The elements that are typically included in expression vectors also include a replicon that functions in E. coli, a gene encoding antibiotic resistance to permit selection of bacteria that harbor recombinant plasmids, and unique restriction sites in nonessential regions of the plasmid to allow insertion of eukaryotic sequences. The particular antibiotic resistance gene chosen is not critical, any of the many resistance genes known in the art are suitable. The prokaryotic sequences are preferably chosen such that they do not interfere with the replication of the DNA in eukaryotic cells, if necessary.

Standard transfection methods are used to produce bacterial, mammalian, yeast or insect cell lines that express large quantities of endopeptidase, which are then purified using standard techniques (see, e.g., Colley et al., J. Biol. Chem. 264:17619-17622 (1989); Guide to Protein Purification, in Methods in Enzymology, vol. 182 (Deutscher, ed., 1990)). Transformation of eukaryotic and prokaryotic cells are performed according to standard techniques (see, e.g., Morrison, J. Bact. 132:349-351 (1977); Clark-Curtiss & Curtiss, Methods in Enzymology 101:347-362 (Wu et al., eds, 1983).

Any of the well-known procedures for introducing foreign nucleotide sequences into host cells may be used. These include the use of calcium phosphate transfection, polybrene, protoplast fusion, electroporation, biolistics, liposomes, microinjection, plasma vectors, viral vectors and any of the other well known methods for introducing cloned genomic DNA, cDNA, synthetic DNA or other foreign genetic material into a host cell (see, e.g., Sambrook et al., supra). It is only necessary that the particular genetic engineering procedure used be capable of successfully introducing at least one gene into the host cell capable of expressing the endopeptidase.

After the expression vector is introduced into the cells, the transfected cells are cultured under conditions favoring expression of the endopeptidase, which is recovered from the culture using standard techniques identified below.

Purification of Endopeptidases

Recombinant endopeptidases can be purified from any suitable expression system by standard techniques, including selective precipitation with such substances as ammonium sulfate; column chromatography, immunopurification methods, and others (see, e.g., Scopes, Protein Purification: Principles and Practice (1982); U.S. Pat. No. 4,673,641; Ausubel et al., supra; and Sambrook et al., supra).

Recombinant proteins are expressed by transformed bacteria in large amounts, typically after promoter induction; but expression can be constitutive. Promoter induction with IPTG is one example of an inducible promoter system. Bacteria are grown according to standard procedures in the art. Fresh or frozen bacteria cells are used for isolation of protein.

Proteins expressed in bacteria may form insoluble aggregates (“inclusion bodies”). Several protocols are suitable for purification of the endopeptidase inclusion bodies. For example, purification of inclusion bodies typically involves the extraction, separation and/or purification of inclusion bodies by disruption of bacterial cells, e.g., by incubation in a buffer of 50 mM TRIS/HCL pH 7.5, 50 mM NaCl, 5 mM MgCl₂, 1 mM DTT, 0.1 mM ATP, and 1 mM PMSF. The cell suspension can be lysed using 2-3 passages through a French Press, homogenized using a Polytron (Brinkman Instruments) or sonicated on ice. Alternate methods of lysing bacteria are apparent to those of skill in the art (see, e.g., Sambrook et al., supra; Ausubel et al., supra).

If necessary, the inclusion bodies are solubilized, and the lysed cell suspension is typically centrifuged to remove unwanted insoluble matter. Proteins that formed the inclusion bodies may be renatured by dilution or dialysis with a compatible buffer. Suitable solvents include, but are not limited to urea (from about 4 M to about 8 M), formamide (at least about 80%, volume/volume basis), and guanidine hydrochloride (from about 4 M to about 8 M). Some solvents which are capable of solubilizing aggregate-forming proteins, for example SDS (sodium dodecyl sulfate), 70% formic acid, are inappropriate for use in this procedure due to the possibility of irreversible denaturation of the proteins, accompanied by a lack of immunogenicity and/or activity. Although guanidine hydrochloride and similar agents are denaturants, this denaturation is not irreversible and renaturation may occur upon removal (by dialysis, for example) or dilution of the denaturant, allowing re-formation of immunologically and/or biologically active protein. Other suitable buffers are known to those skilled in the art. Endopeptidases are separated from other bacterial proteins by standard separation techniques, e.g., with Ni-NTA agarose resin.

Alternatively, it is possible to purify the endopeptidases from the bacteria periplasm. After lysis of the bacteria, when the endopeptidases are exported into the periplasm of the bacteria, the periplasmic fraction of the bacteria can be isolated by cold osmotic shock in addition to other methods known to skill in the art. To isolate recombinant proteins from the periplasm, the bacterial cells are centrifuged to form a pellet. The pellet is resuspended in a buffer containing 20% sucrose. To lyse the cells, the bacteria are centrifuged and the pellet is resuspended in ice-cold 5 mM MgSO₄ and kept in an ice bath for approximately 10 minutes. The cell suspension is centrifuged and the supernatant decanted and saved. The recombinant proteins present in the supernatant can be separated from the host proteins by standard separation techniques well known to those of skill in the art.

Often as an initial step, particularly if the protein mixture is complex, an initial salt fractionation can separate many of the unwanted host cell proteins (or proteins derived from the cell culture media) from the recombinant protein of interest. The preferred salt is ammonium sulfate. Ammonium sulfate precipitates proteins by effectively reducing the amount of water in the protein mixture. Proteins then precipitate on the basis of their solubility. The more hydrophobic a protein is, the more likely it is to precipitate at lower ammonium sulfate concentrations. A typical protocol includes adding saturated ammonium sulfate to a protein solution so that the resultant ammonium sulfate concentration is between 20-30%. This concentration will precipitate the most hydrophobic of proteins. The precipitate is then discarded (unless the protein of interest is hydrophobic) and ammonium sulfate is added to the supernatant to a concentration known to precipitate the protein of interest. The precipitate is then solubilized in buffer and the excess salt removed if necessary, either through dialysis or diafiltration. Other methods that rely on solubility of proteins, such as cold ethanol precipitation, are well known to those of skill in the art and can be used to fractionate complex protein mixtures.

The molecular weight of the endopeptidases can be used to isolate it from proteins of greater and lesser size using ultrafiltration through membranes of different pore size (for example, Amicon or Millipore membranes). As a first step, the protein mixture is ultrafiltered through a membrane with a pore size that has a lower molecular weight cut-off than the molecular weight of the protein of interest. The retentate of the ultrafiltration is then ultrafiltered against a membrane with a molecular cut off greater than the molecular weight of the protein of interest. The recombinant protein will pass through the membrane into the filtrate. The filtrate can then be chromatographed as described below.

The endopeptidases can also be separated from other proteins on the basis of its size, net surface charge, hydrophobicity, and affinity for ligands. In addition, antibodies raised against proteins can be conjugated to column matrices and the proteins immunopurified. All of these methods are well known in the art. It will be apparent to one of skill that chromatographic techniques can be performed at any scale and using equipment from many different manufacturers (e.g., Pharmacia Biotech).

Nucleic Acids

In another aspect, the present invention provides an isolated nucleic acid encoding a endopeptidase which site-specifically cleaves a peptide bond of a post-translationally modified polypeptide at a site of post-translational modification and which comprises one or more point mutations at one or more amino acid positions within the endopeptidase active site.

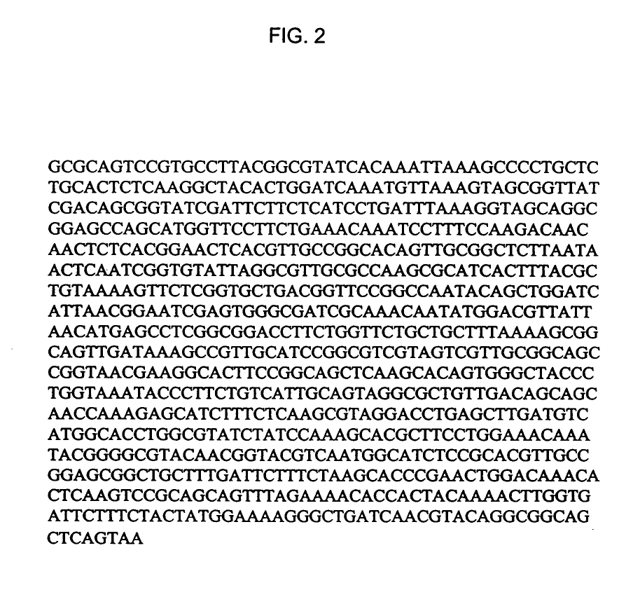

In another exemplary embodiment, the isolated nucleic acid hybridizes under highly stringent hybridization conditions to a nucleic acid sequence of FIG. 2, wherein the hybridization reaction is incubated at 42° C. in a solution comprising 50% formamide, 5×SSC and 1% SDS, and washed at 65° C. in a solution comprising 0.2×SSC and 0.1% SDS. In an exemplary embodiment, the nucleic acid also encodes an endopeptidase containing at least one amino acid substitution selected from P129G, E156R, S191K, G166K, G127S, E156K, P129K, P129R, S159R, and E156G (see FIG. 1). In another related embodiment, the nucleic acid encodes an endopeptidase containing one of the following combinations of substitution point mutations: G127S and E156R; P129G and E156R; P129G and E156K; E156R and S191K; P129K and E156R; P129R and E156K; E156K and G166K; E156K and S191K; E156K and S191K and S159R; P129R and E156R; and E156G and G166K. In another related embodiment, the nucleic acid encodes an endopeptidase containing one or two amino acid substitutions selected from P129G, E156R, S191K, G166K, and G127S. In another related embodiment, the nucleic acid to which the isolated nucleic acid hybridizes under highly stringent hybridization conditions additionally contains a nucleic acid sequence encoding a signal sequence as shown in FIG. 8 (in bold). In another related embodiment, the nucleic acid to which the isolated nucleic acid hybridizes under highly stringent hybridization conditions additionally contains a nucleic acid sequence encoding a prodomain as shown in FIG. 8 (underlined).

In an exemplary embodiment, the isolated nucleic acid contains a subsequence having at least 70% nucleic acid sequence identity to a nucleic acid sequence of FIG. 2. In a related embodiment, the nucleic acid has 75%, 76%, 77%, 78%, 79%, 80%, 85%. 86%, 87%, 88%, 89%, 90%, 91%, 92%, 93%, 94%, 95%, 96%, 97%, or 98% amino acid sequence identity. In a related embodiment, the nucleic acid to which the isolated nucleic acid hybridizes under highly stringent hybridization conditions additionally contains a nucleic acid sequence encoding a signal sequence as shown in FIG. 8 (in bold). In another related embodiment, the nucleic acid to which the isolated nucleic acid hybridizes under highly stringent hybridization conditions additionally contains a nucleic acid sequence encoding a prodomain as shown in FIG. 8 (underlined).

In another related embodiment, the nucleic acid contains a subsequence as described above and encodes an endopeptidase containing a subsequence as described above and contains at least one amino acid substitution selected from P129G, E156R, S191K, G166K, G127S, E156K, P129K, P129R, S159R, and E156G (see FIG. 1). In another related embodiment, the nucleic acid contains a subsequence as described above and encodes an endopeptidase containing one of the following combinations of substitution point mutations: G127S and E156R; P129G and E156R; P129G and E156K; E156R and S191K; P129K and E156R; P129R and E156K; E156K and G166K; E156K and S191K; E156K and S191K and S159R; P129R and E156R; and E156G and G166K. In another related embodiment, the nucleic acid contains a subsequence as described above and encodes an endopeptidase containing one or two amino acid substitutions selected from P129G, E156R, S191K, G166K, and G127S.

The present invention also provides expression vectors containing the above nucleic acids and host cells transfected with the vectors.

Informatics

The methods described above will produce valuable data regarding the location of post-translational modifications on polypeptides. The data may be provided in a variety of dataset forms, such as polypeptide fragment sequences, polypeptide fragmentation patterns (as deduced, for example, from two dimensional gel electrophoresis or mass spectrometry), polypeptide fragment elution patterns (for example, from HPLC columns or capillary gel electrophoresis columns), and the like. Thus, the site of post-translational modification can be determined, for example, by comparing the mass spectral or two-dimensional electrophoretic fragmentation pattern of a degraded post-translationally modified polypeptide using informatic techniques. In an exemplary embodiment, the informatic technique includes comparing the mass spectral or two-dimensional electrophoretic fragmentation pattern of a degraded post-translationally modified polypeptide to a known or predicted fragmentation pattern of the polypeptide using the methodologies disclosed below.

As high-resolution, high-sensitivity datasets acquired using the methods of the invention become available to the art, significant progress in the areas of diagnostics, therapeutics, drug development, biosensor development, and other related areas will occur. For example, disease markers can be identified and utilized for better confirmation of a disease condition or stage (see, U.S. Pat. Nos. 5,672,480; 5,599,677; 5,939,533; and 5,710,007). Subcellular toxicological information can be generated to better direct drug structure and activity correlation (see, Anderson, L., “Pharmaceutical Proteomics: Targets, Mechanism, and Function,” paper presented at the IBC Proteomics conference, Coronado, Calif. (Jun. 11-12, 1998)). Subcellular toxicological information can also be utilized in a biological sensor device to predict the likely toxicological effect of chemical exposures and likely tolerable exposure thresholds (see, U.S. Pat. No. 5,811,231). Similar advantages accrue from datasets relevant to other biomolecules and bioactive agents (e.g., nucleic acids, saccharides, lipids, drugs, and the like).

Thus, in an exemplary embodiment, the present invention provides a database that includes at least one set of data assay data. The data contained in the database is acquired using a method of the invention. The database can be in substantially any form in which data can be maintained and transmitted, but is preferably an electronic database. The electronic database of the invention can be maintained on any electronic device allowing for the storage of and access to the database, such as a personal computer, but is preferably distributed on a wide area network, such as the World Wide Web.

The compositions and methods described herein may be used to identify sites of post-translational modifications, or a lack thereof, on a variety of polypeptides from a divers array of sources. Such methods provide an abundance of information, which can be correlated with pathological conditions, predisposition to disease, drug testing, therapeutic monitoring, gene-disease causal linkages, identification of correlates of immunity and physiological status, among others. Although the data generated from the methods of the invention is suited for manual review and analysis, in an exemplary embodiment, prior data processing using high-speed computers is utilized.

An array of methods for indexing and retrieving biomolecular information is known in the art. For example, U.S. Pat. Nos. 6,023,659 and 5,966,712 disclose a relational database system for storing biomolecular sequence information in a manner that allows sequences to be catalogued and searched according to one or more protein function hierarchies. U.S. Pat. No. 5,953,727 discloses a relational database having sequence records containing information in a format that allows a collection of partial-length DNA sequences to be catalogued and searched according to association with one or more sequencing projects for obtaining full-length sequences from the collection of partial length sequences. U.S. Pat. No. 5,706,498 discloses a gene database retrieval system for making a retrieval of a gene sequence similar to a sequence data item in a gene database based on the degree of similarity between a key sequence and a target sequence. U.S. Pat. No. 5,538,897 discloses a method using mass spectroscopy fragmentation patterns of peptides to identify amino acid sequences in computer databases by comparison of predicted mass spectra with experimentally-derived mass spectra using a closeness-of-fit measure. U.S. Pat. No. 5,926,818 discloses a multi-dimensional database comprising a functionality for multi-dimensional data analysis described as on-line analytical processing (OLAP), which entails the consolidation of projected and actual data according to more than one consolidation path or dimension. U.S. Pat. No. 5,295,261 reports a hybrid database structure in which the fields of each database record are divided into two classes, navigational and informational data, with navigational fields stored in a hierarchical topological map which can be viewed as a tree structure or as the merger of two or more such tree structures.

The present invention provides a computer database comprising a computer and software for storing in computer-retrievable form assay data records cross-tabulated, for example, with data specifying the source of the post translationally modified polypeptide and/or the host cell or organism from which each sequence specificity record was obtained.

In an exemplary embodiment, at least one of the sources of the post translationally modified polypeptide is from a tissue sample known to be free of pathological disorders. In a variation, at least one of the sources is a known pathological tissue specimen, for example, a neoplastic lesion or a tissue specimen containing a pathogen such as a virus, bacteria or the like. In another variation, the assay records cross-tabulate one or more of the following parameters for each target species in a sample: (1) a unique identification code, which can include, for example, a target molecular structure and/or characteristic separation coordinate (e.g., electrophoretic coordinates); (2) sample source; and (3) absolute and/or relative quantity of the target species present in the sample.

The invention also provides for the storage and retrieval of a collection of target data in a computer data storage apparatus, which can include magnetic disks, optical disks, magneto-optical disks, DRAM, SRAM, SGRAM, SDRAM, RDRAM, DDR RAM, magnetic bubble memory devices, and other data storage devices, including CPU registers and on-CPU data storage arrays. Typically, the data records are stored as a bit pattern in an array of magnetic domains on a magnetizable medium or as an array of charge states or transistor gate states, such as an array of cells in a DRAM device (e.g., each cell comprised of a transistor and a charge storage area, which may be on the transistor). In one embodiment, the invention provides such storage devices, and computer systems built therewith, comprising a bit pattern encoding a protein expression fingerprint record comprising unique identifiers for at least 10 polypeptide data records cross-tabulated with polypeptide sources.

In an exemplary embodiment, the invention provides a method for identifying post-translationally modified sites, or a lack thereof, on related polypeptides, comprising performing a computerized comparison between a polypeptide sequence assay record stored in or retrieved from a computer storage device or database and at least one other sequence. The comparison can include a sequence analysis or comparison algorithm or computer program embodiment thereof (e.g., FASTA, TFASTA, GAP, BESTFIT) and/or the comparison may be of the relative amount of a polypeptide sequence in a pool of sequences determined from a polypeptide sample.

The invention also preferably provides a magnetic disk, such as an IBM-compatible (DOS, Windows, Windows95/98/2000, Windows NT, OS/2) or other format (e.g., Linux, SunOS, Solaris, AIX, SCO Unix, VMS, MV, Macintosh, etc.) floppy diskette or hard (fixed, Winchester) disk drive, comprising a bit pattern encoding data from an assay of the invention in a file format suitable for retrieval and processing in a computerized sequence analysis, comparison, or relative quantitation method.

The invention also provides a network, comprising a plurality of computing devices linked via a data link, such as an Ethernet cable (coax or 10BaseT), telephone line, ISDN line, wireless network, optical fiber, or other suitable signal transmission medium, whereby at least one network device (e.g., computer, disk array, etc.) comprises a pattern of magnetic domains (e.g., magnetic disk) and/or charge domains (e.g., an array of DRAM cells) composing a bit pattern encoding data acquired from an assay of the invention.

The invention also provides a method for transmitting assay data that includes generating an electronic signal on an electronic communications device, such as a modem, ISDN terminal adapter, DSL, cable modem, ATM switch, or the like, wherein the signal includes (in native or encrypted format) a bit pattern encoding data from an assay or a database comprising a plurality of assay results obtained by the method of the invention.

In an exemplary embodiment, the invention provides a computer system for comparing a post translationally modified polypeptide to a database containing an array of data structures, such as an assay result obtained by the method of the invention, and ranking database polypeptide targets based on the degree of identity and gap weight to the target data. A central processor is preferably initialized to load and execute the computer program for alignment and/or comparison of the assay results. Data for a polypeptide target is entered into the central processor via an I/O device. Execution of the computer program results in the central processor retrieving the assay data from the data file, which comprises a binary description of an assay result.

The data or record and the computer program can be transferred to secondary memory, which is typically random access memory (e.g., DRAM, SRAM, SGRAM, or SDRAM). The polypeptide targets are ranked according to the degree of correspondence between a selected assay characteristic (e.g., binding to a selected binding functionality) and the same characteristic of the post translationally modified polypeptide target and results are output via an I/O device. For example, a central processor can be a conventional computer (e.g., Intel Pentium, PowerPC, Alpha, PA-8000, SPARC, MIPS 4400, MIPS 10000, VAX, etc.); a program can be a commercial or public domain molecular biology software package (e.g., UWGCG Sequence Analysis Software, Darwin); a data file can be an optical or magnetic disk, a data server, a memory device (e.g., DRAM, SRAM, SGRAM, SDRAM, EPROM, bubble memory, flash memory, etc.); an I/O device can be a terminal comprising a video display and a keyboard, a modem, an ISDN terminal adapter, an Ethernet port, a punched card reader, a magnetic strip reader, or other suitable I/O device.

The invention also preferably provides the use of a computer system, such as that described above, which comprises: (1) a computer; (2) a stored bit pattern encoding a collection of peptide sequence specificity records obtained by the methods of the invention, which may be stored in the computer; (3) a comparison post translationally modified polypeptide target; and (4) a program for alignment and comparison, typically with rank-ordering of comparison results on the basis of computed similarity values.

Kits

The present invention also provides a kit for practicing a method set forth herein. In an exemplary embodiment, the kit includes one or more component useful to practice the method of the invention and instructions for using that component to practice the method of the invention.

In a preferred embodiment, the kit includes a container of an endopeptidase for the present invention and instructions for using the endopeptidase to determine sites of post-translationally modification on the polypeptide. The examples that follow are intended to further illustrate the invention not to limit the scope of the invention.

The terms and expressions which have been employed herein are used as terms of description and not of limitation, and there is no intention in the use of such terms and expressions of excluding equivalents of the features shown and described, or portions thereof, it being recognized that various modifications are possible within the scope of the invention claimed. Moreover, any one or more features of any embodiment of the invention may be combined with any one or more other features of any other embodiment of the invention, without departing from the scope of the invention. For example, the endonucleases described in the endonuclease section are equally applicable to the informatics methods described herein. All publications, patents, and patent applications cited herein are hereby incorporated by reference in their entirety for all purposes.

### EXAMPLES

Materials

The BG2036 protease deficient strain of B. subtilis and the pSS5 shuttle vector containing the subtilisin BPN′ gene were employed. All pNA tetrapeptide substrates were from Bachem or Sigma. All Fmoc amino acids were from Bachem, Novabiochem or Advanced Chemtech. All other reagents were from Sigma unless noted.

### Example 1

Example 1 describes a method for identifying candidate amino acid positions in a model endopeptidase by comparing the three-dimensional structures of the model endopeptidase and a post-transitionally modified polypeptide. In addition, the structure of potential candidate endopeptidases are compared with a post-transitionally modified polypeptide. In this example, the post-translationally modified polypeptide is a phosphotyrosine polypeptide and the model endopeptidase is a subtilisin containing a sub-sequence of FIG. 7.

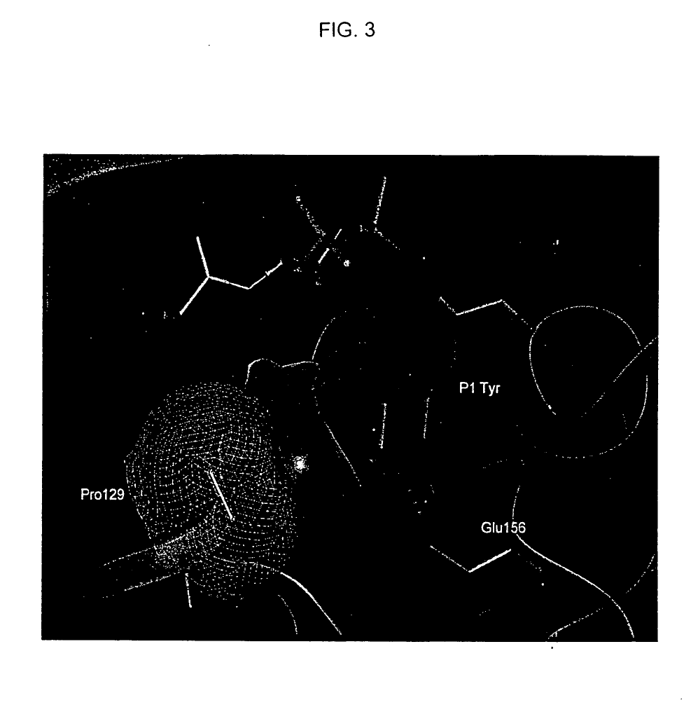

Candidate amino acid positions were identified by comparing the three-dimensional model of a phosphotyrosine polypeptide and the subtilisin endopeptidase (see FIG. 3). As seen in FIG. 3, the phosphotyrosine moiety sterically clashes with proline 129 (mesh) and unfavorably interacts with glutamate 156. Three-dimensional models of potential candidate subtilisin endopeptidases were also generated to assess the ability of various amino acids to bind to the phosphotyrosine polypeptide when introduced into the candidate amino acid positions. The subtilisin endopeptidase model was based on the known crystal structure. The phosphotyrosine polypeptide structure was predicted based on the primary sequence. The structures of subtilisin, the potential candidate subtilisins and the phosphotyrosine substrates were built using the biopolymer function within the InsightII software package starting from the PDB file 1 SUA on a Silicon Graphics O₂ workstation. Backbone atoms were left fixed and reasonable side chain rotamers were evaluated using the Bump function to check for intermolecular and intramolecular steric clashes.

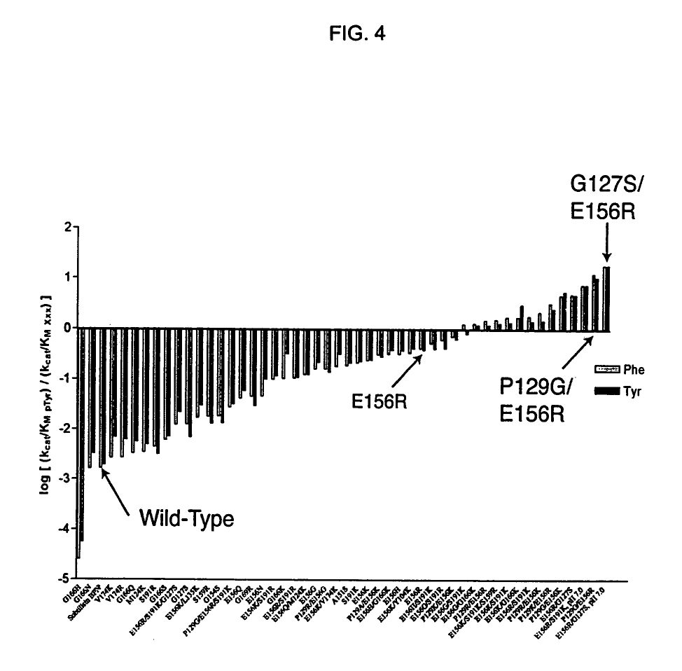

The substitution point mutations of the resulting candidate subtilisins are shown in the abscissa of the graph of FIG. 4.

### Example 2

Example 2 describes methods of constructing and purifying exemplary candidate endopeptidases. The candidate endopeptidases in this example are derived from the subtilisin model endopeptidase as described in Example 1.

Substitution point mutations as shown in FIG. 4 were introduced into the subtilisin gene in the pSS5 vector using the Quikchange protocol for PCR mutagenesis (Stratagene). All mutations were confirmed by dideoxy sequencing. Monomer plasmid DNA was transformed into a RecA+ strain of E. coli (JM101, Stratagene) to prepare multimeric plasmids. This plasmid DNA was used to transform a protease deficient strain (BG2036) of B. subtilis (Kunst, 1993). Transformants were selected with 12.5 μg/ml chloramphenicol and restreaked on 1% skim milk plates to confirm protease activity.

Subtilisin candidate endopeptidases were purified essentially by the method of Estell. In brief, 500 ml 2×YT (12.5 ug/ml chloramphenicol) was inoculated with 5 ml of an overnight culture and allowed to grow for 24 hours at 37° C. Cells were pelleted by centrifugation and one equivalent (500 ml) of −20° C. ethanol was added to the supernatant. The supernatant was centrifuged for 15 minutes at 8000 rpm, and the pellet was discarded. A second equivalent of −20° C. ethanol (1000 ml) was added to the supernatant, which was then left overnight at −20° C. The resulting supernatant was centrifuged for 15 minutes at 5000 rpm and the supernatant was discarded. The pellet was resuspended in a minimal volume (2 to 3 ml) of 50 mM Tris, pH 8.0, 5 mM CaCl₂ and clarified at 18,000 g for 30 minutes. The supernatant was then removed and precipitated overnight at 4° C. with 3.5 volumes of 90% saturated ammonium sulphate. The ammonium sulphate pellet was collected by centrifugation and resuspended in 2-3 ml of 25 mM MES, 5 mM CaCl₂, pH 5.5 and dialyzed at 4° C. in the same buffer for 24 hours (3×1 L). At this stage, protein preparations were typically >75% pure as judged by SDS-PAGE. For many mutants, the dialyate was then loaded onto a Mono S column attached to a Biocad FPLC and eluted using the same buffer with a gradient 0-500 mM NaCl. Fractions were collected, aliquoted, flash frozen in liquid nitrogen and stored at −80 C.

### Example 3

Example 3 describes the synthesis of a series of test polypeptides. In this example, the test polypeptides comprise a fluorescent donor-quencher pair.

Test polypeptides were synthesized using standard Fmoc peptide synthesis protocols starting from Wang resin preloaded with Fmoc-Asp(O-tBu). For the sulphotyrosine peptide, a 2-chlorotrityl resin was utilized combined with a low temperature cleavage and deprotection (10 hours at 0 C.) to overcome the inherent acid lability of the tyrosine sulphate. All peptides were purified to >95% by reverse phase HPLC utilizing an acetonitrile/water/0.1% TFA solvent system and characterized by electrospray MS on a Perkin Elmer mass spectrometer.

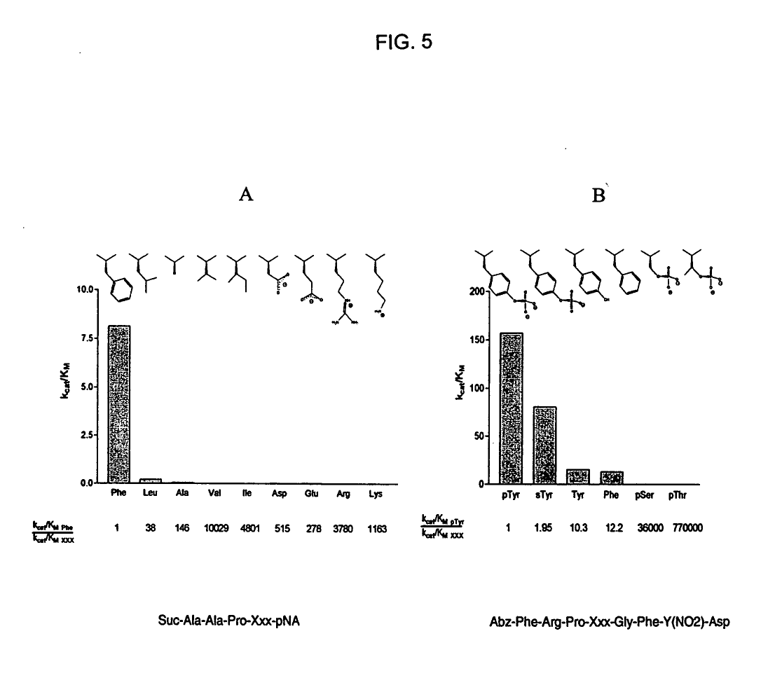

The resulting test polypeptides are shown in FIG. 5, wherein Xxx represents a phosphotyrosine, sulfonyl tyrosine, tyrosine, phenylalanine, phosphoserine, phosphothreonine, alanine, valine, leucine, isoleucine, aspartic acid, glutamic acid, arginine, or lysine as shown. The data in panel A was obtained using a test polypeptide containing a succinyl-paranitroanalide fluorogenic donor-acceptor pair. The data in panel B was obtained using a test polypeptide containing am aminobenzoic acid-tyrosine(NO₂)-aspartic acid fluorogenic donor-acceptor pair.

### Example 4

Example 4 demonstrates a method for identifying an endopeptidase that site-specifically cleaves a peptide bond of a post-translationally modified polypeptide. The methods involve assaying the candidate subtilisins of Example 2 with the test polypeptides of Example 3.

Kinetics for the fluorogenic substrates of the series Abz-Phe-Arg-Pro-Xxx-Gly-Phe-Y(NO₂)-Asp were measured in 50 mM Bicine, 2 mM CaCl₂, pH 8.5 at 25° C. by monitoring fluorescence at 420 nm upon excitation at 320 nm using a instrument. Initial rate data from 8 substrate concentrations bracketing the K_{M} was measured in triplicate and fit directly to the Michaelis Menten equation using the Prism software package (GraphPad,). When it was not possible to saturate the enzyme, values for k_{cat}/K_{M} were obtained from initial rates at low concentrations (10[S]<K_{M}) using the relationship k_{cat}/K_{M}=V₀[S]. Kinetics for tetrapeptide substrates of the series Suc-Ala-Ala-Pro-Xxx-pNa were measured by monitoring the change in absorbance at 412 nm over time using a Uvikon spectrophotometer. Protein concentrations were determined spectrophotometrically using an extinction coefficient of 32.2 mM⁻¹ cm⁻¹ at 280 nm (Matsubara, 1965).

### Example 5

Example 5 demonstrates that subtilisin endopeptidases that site-specifically cleave a phosphotyrosine polypeptide at the phosphorylated tyrosine are obtained using the methods of the present invention, as demonstrated in Examples 1-4.

FIG. 4 illustrates the phosphotyrosine site-specificity of the candidate subtilisin endopeptidases and the model subtilisin endopeptidase against either an unmodified tyrosine or phenylalanine. As shown in FIG. 4, subtilisin endopeptidases containing the following substitution point mutations were found to preferably cleave at the phosphotyrosine residue over a tyrosine residue or phenylalanine residue: G127S and E156R, P129G and E156R, P129G and E156K, E156R and S191K, P129K and E156R, P129R and E156K, E156K and G166K, E156K and S191K, E156K and S191K and S159R, P129R and E156R, and E156G and G166K.

FIG. 5 shows kinetic data for the site-specific cleavage at a phosphotyrosine by a subtilisin endopeptidase containing the substitution point mutations P129G and E156R.

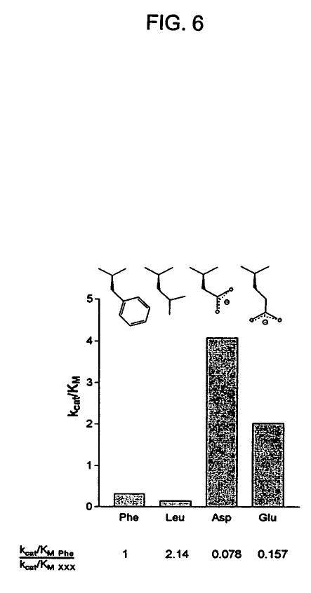

FIG. 6 shows kinetic data for the site-specific cleavage at a phosphotyrosine by a subtilisin endopeptidase containing the substitution point mutations G127S and E156R.

What is claimed is:

1. A method for mapping a site of post-translational modification on a post-translationally modified polypeptide, said method comprising:
(a) site-specifically cleaving a peptide bond of the post-translationally modified polypeptide with an endopeptidase at said site of post-translational modification to produce a degraded post-translationally modified polypeptide; and
(b) after step (a), determining said site of post-translational modification.

2. The method of claim 1, wherein said post-translational modification is selected from phosphorylation, sulfonation, glycosylation, acetylation, methylations, ADP-ribosylation, methionine oxidation, cysteine oxidation, and cysteine lipidation.

3. The method of claim 1, wherein said post-translational modification is phosphorylation of an amino acid selected from tyrosine, serine, and threonine.

4. The method of claim 1, wherein said post-translational modification is sulfonation of a tyrosine.

5. The method of claim 1, wherein said site of post-translational modification is determined by a method comprising determining the mass spectrometry fragmentation pattern of the degraded post-translationally modified polypeptide.

6. The method of claim 1, wherein said endopeptidase is a serine protease comprising an active site that specifically binds to said post-translational modification.

7. The method of claim 6, wherein said serine protease is subtilisin.

8. A serine protease which site-specifically cleaves a peptide bond of a post-translationally modified polypeptide at a site of post-translational modification, wherein said serine protease comprises an active site that binds to said site of post-translational modification.

9. The serine protease of claim 8, wherein said post-translational modification is selected from phosphorylation, sulfonation, glycosylation, and acetylation.

10. The serine protease of claim 8, wherein said post-translational modification is phosphorylation of an amino acid selected from tyrosine, serine, and threonine.

11. The serine protease of claim 8, wherein said post-translational modification is sulfonation of a tyrosine.

12. The serine protease of claim 8, wherein said serine protease is subtilisin.

13. The serine protease of claim 8, wherein said serine protease is encoded by a nucleic acid sequence that hybridizes under highly stringent hybridization conditions to a nucleic acid encoding a polypeptide comprising an amino acid sequence of FIG. 1, wherein the hybridization reaction is incubated at 42° C. in a solution comprising 50% formamide, 5×SSC and 1% SDS, and washed at 65° C. in a solution comprising 0.2×SSC and 0.1% SDS.

14. The serine protease of claim 8, wherein said serine protease comprises a subsequence having at least 70% amino acid sequence identity to an amino acid sequence of FIG. 1.

15. The serine protease of claim 8, wherein said serine protease comprises a subsequence having at least 70% amino acid sequence identity to an amino acid sequence of FIG. 1 and contains at least one amino acid substitution selected from P129G, E156R, S191K, G166K, and G127S.

16. The serine protease of claim 8, wherein said serine protease is encoded by an expression vector.

17. A host cell comprising the expression vector of claim 16.

18. An endopeptidase that site-specifically cleaves a peptide bond of a post-translationally modified polypeptide at a site of post-translational modification, said endopeptidase produced by a method comprising:
(a) introducing one or more point mutations to a model endopeptidase at one or more candidate amino acid positions in an active site of said model endopeptidase to produce a plurality of candidate endopeptidases, wherein at least one of said plurality of candidate endopeptidases is an endopeptidase that site-specifically cleaves a peptide bond of a post-translationally modified polypeptide at a site of post-translational modification; and
(b) identifying said endopeptidase that site-specifically said peptide bond of said post-translationally modified polypeptide cleaves at said site of post-translational modification.

19. The endopeptidase of claim 18, wherein said model endopeptidase comprises a subsequence having at least 70% amino acid sequence identity to an amino acid sequence of FIG. 1.

20. The endopeptidase of claim 18, wherein said model endopeptidase is encoded by a nucleic acid sequence that hybridizes under highly stringent hybridization conditions to a nucleic acid encoding a polypeptide comprising an amino acid sequence of FIG. 1, wherein the hybridization reaction is incubated at 42° C. in a solution comprising 50% formamide, 5×SSC and 1% SDS, and washed at 65° C. in a solution comprising 0.2×SSC and 0.1% SDS.

21. The endopeptidase of claim 18, wherein said one or more candidate amino acid positions is selected from P129, E156, S191, G166, and G127.

22. The endopeptidase of claim 18, wherein said one or more candidate amino acid positions is P129 and said point mutation is a glycine or alanine substitution.

23. The endopeptidase of claim 18, wherein said one or more candidate amino acid positions is E156 and said point mutation is an arginine substitution.

24. The endopeptidase of claim 18, wherein said one or more candidate amino acid positions is E156 and said point mutation is a lysine substitution.

25. The endopeptidase of claim 18, wherein said one or more candidate amino acid positions is P129 and E156, wherein said point mutation is glycine at p129 and arginine at E156.

26. The endopeptidase of claim 18, wherein, before step (a), said one or more candidate amino acid positions are identified by a method comprising:
(i) generating a three-dimensional structure of said model endopeptidase active site;
(ii) generating a three-dimensional structure of said post-translationally modified polypeptide;
(iv) comparing the three-dimensional structure of said model endopeptidase active site with said post-translationally modified polypeptide, thereby identifying one or more candidate amino acid positions that, upon introduction of one or more point mutations at one or more of said candidate amino acid positions, produces a plurality of candidate endopeptidases, wherein at least one of said plurality of candidate endopeptidases is said endopeptidase that site-specifically said peptide bond of said post-translationally modified polypeptide cleaves at said site of post-translational modification.

27. An isolated nucleic acid encoding a endopeptidase which site-specifically cleaves a peptide bond of a post-translationally modified polypeptide at a site of post-translational modification and which comprises one or more point mutations at one or more amino acid positions within the endopeptidase active site,
wherein said isolated nucleic acid hybridizes under highly stringent hybridization conditions to a nucleic acid sequence of FIG. 2, wherein the hybridization reaction is incubated at 42° C. in a solution comprising 50% formamide, 5×SSC and 1% SDS, and washed at 65° C. in a solution comprising 0.2×SSC and 0.1% SDS.

28. An expression vector comprising the nucleic acid of claim 27.

29. A host cell transfected with the vector of claim 28.

30. An isolated nucleic acid encoding a endopeptidase which site-specifically cleaves a polypeptide backbone amide bond of a post-translationally modified polypeptide at a site of post-translational modification and which comprises one or more point mutations at one or more amino acid positions within the endopeptidase active site,
wherein said isolated nucleic acid comprises a subsequence having at least 70% nucleic acid sequence identity to a nucleic acid sequence of FIG. 2.

31. An expression vector comprising the nucleic acid of claim 30.

32. A host cell transfected with the vector of claim 30.
